(19) United States
(12) Patent Application Publication
(10) Pub. No.: US 2003/0114442 A1
(43) Pub. Date: Jun. 19, 2003

(54) Substituted piperazine derivatives as mtp inhibitors

(76) Inventors: Armin Heckel, Biberach (DE); Thorsten Lehmann-Lintz, Ochsenhausen/Laubach (DE); Leo Thomas, Biberach (DE); Michael Mark, Biberach (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/168,926

(22) PCT Filed: Dec. 16, 2000
(86) PCT No.: PCT/EP00/12842

(30) Foreign Application Priority Data
Dec. 27, 1999 (DE) 199 63 235.9

(51) Int. Cl. A61K 31/551; A61K 31/496; A61K 31/495; C 07D 4 5/02; C 07D 4 3/02

(52) U.S. Cl. 514/218; 514/253.03; 514/254.11; 514/255.01; 514/255.02; 540/575; 544/361; 544/375; 544/380

(57) ABSTRACT

The present invention relates to substituted piperazine derivatives of general formula

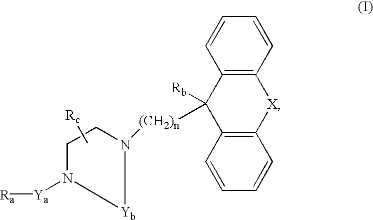

wherein

Rₐ to R_{c}, Yₐ, Y_{b}, X and n are defined as in claim 1, the isomers and salts thereof, particularly the physiologically acceptable salts thereof, which are valuable inhibitors of the microsomal triglyceride-transfer protein (MTP), medicaments containing these compounds and their use, as well as the preparation thereof.

The present invention relates to substituted piperazine derivatives of general formula

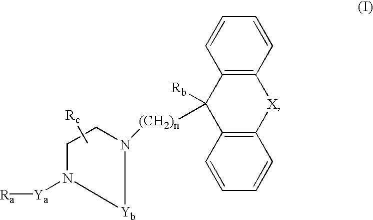

their isomers, their salts, particularly the physiologically acceptable salts thereof which have valuable pharmacological properties.

The compounds of the above general formula I are valuable inhibitors of the microsomal triglyceride-transfer protein (MTP) and are therefore suitable for lowering the plasma level of the atherogenic lipoproteins.

In the above general formula I

n denotes the number 2, 3, 4 or 5,

X denotes a carbon-carbon bond, an oxygen atom, a methylene, ethylene, imino or N-(C₁₋₃-alkyl)-imino group,

Yₐ denotes a carbonyl or sulphonyl group,

Y_{b} denotes the group —(CH₂)ₘ—, wherein m denotes the number 2 or 3 and wherein a hydrogen atom may be replaced by a C₁₋₃-alkyl group or a methylene group linked to a nitrogen atom may be replaced by a carbonyl group,

Rₐ denotes a C₁₋₆-alkoxy-, phenyl-C₁₋₃-alkoxy or amino group, wherein the amino group may be mono- or disubstituted by C₁₋₃-alkyl-, phenyl-C₁₋₄-alkyl or phenyl groups and the substituents may be identical or different,

a phenyl-, naphthyl, tetrahydronaphthyl, phenoxy or heteroaryl group, a C₁₋₉-alkyl group optionally substituted by a hydroxy, C₁₋₃-alkoxy, C₁₋₄-alkoxycarbonyl or C₁₋₄-alkyl-carbonyloxy group, which may be substituted in the alkyl moiety by a C₁₋₃-alkyl group, by one or two phenyl groups, by a naphthyl, fluorenyl, phenoxy, heteroaryl or C₃₋₇-cycloalkyl group, or a C₃₋₇-cycloalkyl group substituted by a phenyl group,

a phenylcarbonyl, naphthylcarbonyl, tetrahydronaphthylcarbonyl, phenoxycarbonyl or heteroarylcarbonyl group, a C₁₋₉-alkylcarbonyl group, which may be substituted in the alkyl moiety by one or two phenyl groups, by a naphthyl, fluorenyl, phenoxy, heteroaryl or C₃₋₇-cycloalkyl group, or a C₃₋₇-cycloalkylcarbonyl group substituted by a phenyl group,

wherein all the phenyl, naphthyl and heteroaryl moieties mentioned under Rₐ hereinbefore may be substituted by the groups R₁ and R₂, wherein

R₁ denotes a hydrogen, fluorine, chlorine or bromine atom, a cyano, C₁₋₃-alkyl, C₂₋₄-alkenyl, phenyl, hydroxy, C₁₋₄-alkoxy, phenyl-C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl, nitro, amino, C₁₋₃-alkylamino, di-(C₁₋₃-alkyl)-amino, phenyl-C₁₋₃-alkylamino, N-(C₁₋₃-alkyl)-phenyl-C₁₋₃-alkylamino, C₁₋₃-alkylcarbonylamino, N-(C₁₋₃-alkyl) —C₁₋₃-alkylcarbonylamino, C₁₋₃-alkylsulphonylamino or N-(C₁₋₃-alkyl)-C₁₋₃-alkylsulphonylamino group and

R₂ denotes a hydrogen, fluorine, chlorine or bromine atom, a C₁₋₃-alkyl, hydroxy or C₁₋₄-alkoxy group, wherein in the abovementioned alkyl and alkoxy moieties of the groups R₁ and R₂ the hydrogen atoms may be wholly or partially replaced by fluorine atoms, or

R₁ and R₂ together denote a methylenedioxy group,

or wherein all the phenyl moieties mentioned above under Rₐ may be substituted by three chlorine or bromine atoms or by three to five fluorine atoms,

R_{b} denotes a carboxy, C₁₋₆-alkoxycarbonyl, C₁₋₆-alkoxycarbonyl-C₁₋₃-alkylcarbonyl, C₃₋₇-cycloalkoxycarbonyl or phenyl-C₁₋₃-alkoxycarbonyl group or a R₃NR₄—CO group wherein

R₃ and R4, which may be identical or different, denote hydrogen atoms, C₁₋₆-alkyl groups wherein the hydrogen atoms may be wholly or partly replaced by fluorine atoms and the C₁₋₃-alkyl moiety of a C₁₋₃-alkylamino group may be substituted by a carboxy or C₁₋₃-alkoxycarbonyl group or in the 2 or 3 position may also be substituted by an amino, C₁₋₃-alkylamino or di-(C₁₋₃-alkyl)-amino group, C₃₋₇-cycloalkyl, pyridyl, pyridinyl-C₁₋₃-alkyl, phenyl, naphthyl or phenyl-C₁₋₃-alkyl groups, wherein the abovementioned phenyl groups may be substituted in each case by a fluorine, chlorine or bromine atom, by a C₁₋₃-alkyl group wherein the hydrogen atoms may be wholly or partly replaced by fluorine atoms, by a hydroxy, C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl or N,N-di-(C₁₋₃-alkyl)-amino group, or

R₃ and R₄ together with the nitrogen atom between them denote a 3- to 7-membered cycloalkyleneimino group, wherein the methylene group in the 4 position of a 6 or 7-membered cycloalkyleneimino group may additionally be replaced by an oxygen or sulphur atom, by a sulphinyl, sulphonyl, imino or N—(C₁₋₃-alkyl)-imino group,

and R_{c} denotes a hydrogen atom or a C₁₋₃-alkyl group,

wherein the tricyclic group in the abovementioned general formula I may additionally be mono- or disubstituted by fluorine or chlorine atoms, by methyl or methoxy groups and the substituents may be identical or different,

by the abovementioned heteroaryl groups is meant a 6-membered heteroaryl group, containing one, two or three nitrogen atoms, or

a 5-membered heteroaryl group, containing an imino group optionally substituted by a C₁₋₃-alkyl group, an oxygen or sulphur atom or

an imino group optionally substituted by a C₁₋₃-alkyl group and one or two nitrogen atoms or

an oxygen or sulphur atom and a nitrogen atom,

wherein a phenyl ring may be fused to the above-mentioned heteroaryl groups via a vinylene group,

and wherein the carboxy group mentioned in the definition of the abovementioned groups may be replaced by a group which can be converted into a carboxy group in vivo or by a group which is negatively charged under physiological conditions.

By a group which may be converted in vivo into a carboxy group is meant, for example, a hydroxymethyl group, a carboxy group esterified with an alcohol wherein the alcoholic moiety is preferably a C₁₋₆-alkanol, a phenyl-C₁₋₃-alkanol, a C₃₋₉-cycloalkanol, wherein a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₁₋₆-alkanoyl group and the cycloalkanol moiety may additionally be substituted by one or two C₁₋₃-alkyl groups, a C₄,-cycloalkenol, a C₃₋₈-alkenol, a phenyl-C₃₋₅-alkenol, a C₃₋₅-alkynol or phenyl-C₃₋₅-alkynol with the proviso that no bond to the oxygen atom starts from a carbon atom which bears a double or triple bond, a C₃₋₈-cycloalkyl-C₁₋₃-alkanol, a bicycloalkanol having a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₐ—CO—O—(R_{b}CR_{c})—OH,

wherein

Rₐ denotes a C₁₋₈-alkyl, C₁₋₇-cycloalkyl, phenyl or phenyl-C₁₋₃-alkyl group,

R_{b} denotes a hydrogen atom, a C₁₋₃-alkyl, C₅₋₇-cycloalkyl or phenyl group and

R_{c} denotes a hydrogen atom or a C₁₋₃-alkyl group,

and by a group which is negatively charged under physiological conditions is meant a carboxy, hydroxysulphonyl, phosphono, tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group.

Moreover, the saturated alkyl and alkoxy moieties which contain more than 2 carbon atoms also include the branched isomers thereof such as, for example, the isopropyl, tert.butyl, isobutyl group, etc.

Preferred compounds of the above general formula I are those wherein

X, Yₐ, Y_{b} and Rₐ to R_{c} are as hereinbefore defined and n denotes the number 3, 4 or 5,

the isomers and salts thereof.

Particularly preferred compounds of the above general formula I are those wherein

n denotes the number 3 or 4,

X denotes a carbon-carbon bond or an oxygen atom,

Yₐ denotes a carbonyl or sulphonyl group,

Y_{b} denotes the group —(CH₂)ₘ, wherein m denotes the number 2 or 3 and wherein a hydrogen atom may be replaced by a C₁₋₃-alkyl group or a methylene group linked to a nitrogen atom may be replaced by a carbonyl group,

Rₐ denotes a C₁₋₄-alkoxy or phenyl-C₁₋₃-alkoxy group,

an amino group monosubstituted by a C₁₋₃-alkyl, phenyl-C₁₋₃-alkyl or phenyl group or disubstituted by a C₁₋₃-alkyl- and a phenyl-C₁₋₃-alkyl or phenyl group, wherein the alkyl moieties may be straight-chain or branched,

a phenyl, naphthyl, 1,2,3,4-tetrahydro-1-naphthyl, 1,2,3,4-tetrahydro-2-naphthyl, phenoxy or heteroaryl group,

a C₁₋₅-alkyl group,

a C₁₋₃-alkyl group substituted by a C₅₋₇-cycloalkyl, phenyl, phenoxy, 1-naphthyl, 2-naphthyl, fluoren-9-yl or heteroaryl group,

a C₁₋₃-alkyl group disubstituted by two phenyl groups or by a phenyl group and a hydroxy, C₁₋₃-alkoxycarbonyl or C₁₋₃-alkyl-carbonyloxy group,

a C₃₋₇-cycloalkyl group substituted by a phenyl group,

a phenylcarbonyl or naphthylcarbonyl group,

wherein all the phenyl moieties mentioned above under Rₐ may be substituted independently of one another by the groups R₁ and R₂ and all the naphthyl and heteroaryl moieties mentioned above under Rₐ may be substituted by the group R₂, wherein

R₁ denotes a hydrogen, fluorine, chlorine or bromine atom, a cyano, C₁₋₃-alkyl, C₃₋₄-alkenyl, phenyl, hydroxy, C₁₋₃-alkoxy, nitro, amino, C₁₋₃-alkylamino, di-(C₁₋₃-alkyl)-amino, C₁₋₃-alkylcarbonylamino or N-(C₁₋₃-alkyl)-C₁₋₃-alkylcarbonylamino group and

R₂ denotes a hydrogen, fluorine, chlorine or bromine atom, a C₁₋₃-alkyl, hydroxy or C₁₋₃-alkoxy group, wherein in the abovementioned alkyl and alkoxy moieties of the groups R₁ and R₂ the hydrogen atoms may be wholly or partially replaced by fluorine atoms, or

R₁ and R₂ together denote a methylenedioxy group,

or wherein all the phenyl moieties mentioned above under Rₐ may be substituted by three chlorine atoms or by three to five fluorine atoms,

R_{b} denotes a C₁₋₃-alkoxycarbonyl, C₁₋₃-alkoxycarbonyl-C₁₋₃-alkylcarbonyl or a R₃NR₄—CO group wherein

R₃ denotes a hydrogen atom or a C₁₋₃-alkyl group and

R₄ denotes a C₁₋₆-alkyl group wherein the hydrogen atoms may be wholly or partly replaced by fluorine atoms, a C₃₋₇-cycloalkyl, phenyl, naphthyl, pyridyl, C₃₋₇-cycloalkyl-C₁₋₃-alkyl, phenyl-C₁₋₃-alkyl or pyridinyl-C₁₋₃-alkyl group,

wherein the abovementioned phenyl groups may be substituted in each case by a fluorine, chlorine or bromine atom, by a C₁₋₃-alkyl group wherein the hydrogen atoms may be wholly or partly replaced by fluorine atoms, or by a hydroxy or C₁₋₃-alkoxy group,

and R_{c} denotes a hydrogen atom or a C₁₋₃-alkyl group,

wherein by the abovementioned heteroaryl group is meant a pyridinyl, pyrazinyl, pyrimidinyl, pyridazinyl, pyrrolyl, furyl, thienyl, oxazolyl, thiazolyl, pyrazolyl, imidazolyl, triazolyl, quinolinyl, quinoxalinyl, quinazolinyl, isoquinolinyl, indolyl or benzimidazolyl group optionally substituted in the carbon skeleton by a C₁₋₃-alkyl group, in which a hydrogen atom bound to a nitrogen atom may be replaced by a C₁₋₃-alkyl group and wherein the 5-membered monocyclic or benzo-condensed heteroaryl groups containing at least one imino group are bound via a carbon or nitrogen atom,

the tricyclic group in the abovementioned general formula I may additionally be substituted by a fluorine or chlorine atom or by a methyl or methoxy group,

and all the abovementioned saturated alkyl and alkoxy moieties which contain more than 2 carbon atoms may be straight-chain or branched, unless stated otherwise,

the isomers and salts thereof.

Most particularly preferred compounds of the above general formula I are those wherein

n denotes the number 4,

X denotes a carbon-carbon bond,

Yₐ denotes a carbonyl group,

Y_{b} denotes the group —(CH₂)₂—,

Rₐ denotes a phenyl-C₁₋₃-alkylamino group,

a straight-chained or branched C₁₋₃-alkyl group substituted by a phenyl or fluoren-9-yl group,

a phenylcarbonyl group,

wherein all the phenyl moieties mentioned above under Ra may be substituted independently of one another by the groups R₁ and R₂, wherein

R₁ denotes a hydrogen, fluorine, chlorine or bromine atom, a cyano or C₁₋₃-alkyl group wherein the hydrogen atoms may be wholly or partly replaced by fluorine atoms, and

R₂ denotes a hydrogen, fluorine, chlorine or bromine atom,

R_{b} denotes a R₃NR₄—CO group wherein

R₃ denotes a hydrogen atom and

R₄ denotes a C₁₋₃-alkyl group wherein the hydrogen atoms may be wholly or partly replaced by fluorine atoms, or a phenyl-C₁₋₃-alkyl group,

wherein the abovementioned phenyl groups may in each case be substituted by a fluorine, chlorine or bromine atom, by a C₁₋₃-alkyl group wherein the hydrogen atoms may be wholly or partly replaced by fluorine atoms, by a hydroxy or C₁₋₃-alkoxy group, and

R_{c} denotes a hydrogen atom or a C₁₋₃-alkyl group,

the isomers and salts thereof.

The following are mentioned as examples of particularly valuable compounds:

(1) 9-[4-(4-phenylacetyl-piperazino)-butyl]-9H-fluorene-9-carboxylic acid-(2,2,2-trifluoro-ethyl)-amide,

(2) 9-(4-{4-[2-(4-trifluoromethyl-phenyl)-acetyl]-piperazino}-butyl)-9H-fluorene-9-carboxylic acid-(2,2,2-trifluoro-ethyl)-amide,

(3) 9-{4-[4-(4-bromo-phenylacetyl)-piperazino]-butyl})-9H-fluorene-9-carboxylic acid-(2,2,2-trifluoro-ethyl)-amide,

(4) 9-{4-[4-(benzylcarbamoyl)-piperazino]-butyl}-9H-fluorene-9-carboxylic acid-(2,2,2-trifluoro-ethyl)-amide,

(5) 9-(4-{4-[2-phenyl-butyryl]-piperazino}-butyl)-9H-fluorene-9-carboxylic acid-(2,2,2-trifluoro-ethyl)-amide,

(6) 9-[4-(4-chlorophenylacetyl-piperazino)-butyl]-9H-fluorene-9-carboxylic acid-(2,2,2-trifluoro-ethyl)-amide,

(7) 9-(4-{4-[(4-fluorophenyl)-acetyl]-piperazino}-butyl)-9H-fluorene-9-carboxylic acid-(2,2,2-trifluoro-ethyl)-amide,

(8) 9-(4-{4-[phenylacetyl]-piperazino}-butyl)-9H-fluorene-9-carboxylic acid-benzyl-amide,

(9) 9-(4-{4-[(3-chlorophenyl)-acetyl]-piperazino)-butyl)-9H-fluorene-9-carboxylic acid-(2,2,2-trifluoro-ethyl)-amide,

(10) 9-(4-{4-[2-oxo-2-phenyl-acetyl]-piperazino}-butyl)-9H-fluorene-9-carboxylic acid-(2,2,2-trifluoro-ethyl)-amide,

(11) 9-(4-{4-[((2,4-dichlorophenyl)-acetyl]-piperazino}-butyl)-9H-fluorene-9-carboxylic acid-(2,2,2-trifluoro-ethyl)-amide,

(12) 9-(4-{4-[(2,3-difluorophenyl)-acetyl]-piperazino}-butyl)-9H-fluorene-9-carboxylic acid-(2,2,2-trifluoro-ethyl)-amide,

(13) 9-(4-{4-[(fluoren-9-yl)-acetyl]-piperazino}-butyl)-9H-fluorene-9-carboxylic acid-(2,2,2-trifluoro-ethyl)-amide,

(14) 9-(4-{4-[(2,4-dichlorophenyl)-acetyl]-(S)-2-methyl-piperazino}-butyl)-9H-fluorene-9-carboxylic acid-(2,2,2-trifluoro-ethyl)-amide and

(15) 9-(4-{4-[(2,4-dichlorophenyl)-acetyl]-(R)-2-methyl-piperazino}-butyl)-9H-fluorene-9-carboxylic acid-(2,2,2-trifluoro-ethyl)-amide,

and the salts thereof.

According to the invention the new compounds are obtained by methods known from the literature, e.g. by the following methods:

a. reacting a compound of general formula

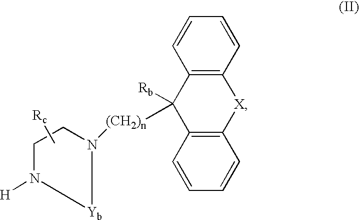

wherein

R_{b}, R_{c}, X, Y_{b} and n are as hereinbefore defined, with a compound of general formula

Rₐ—Yₐ-Z₁, (III)

wherein

Rₐ and Yₐ are as hereinbefore defined and

Z₁ denotes a hydroxy group, a nucleofugic leaving group such as a halogen atom, e.g. a chlorine, bromine or iodine atom, or,

if Yₐ denotes a carbonyl group, Z₁ together with the hydrogen atom of an adjacent NH group in the group Rₐ denotes another carbon-nitrogen bond.

The reaction is optionally carried out in a solvent or mixture of solvents such as methylene chloride, dimethylformamide, benzene, toluene, chlorobenzene, tetrahydrofuran, benzene/tetrahydrofuran or dioxane optionally in the presence of an inorganic or organic base and optionally in the presence of a dehydrating agent, expediently at temperatures between −50 and 150° C., preferably at temperatures between −20 and 80° C.

With a compound of general formula III wherein Z₁ denotes a leaving group, the reaction is optionally carried out in a solvent or mixture of solvents such as methylene chloride, dimethylformamide, benzene, toluene, chlorobenzene, tetrahydrofuran, benzene/tetrahydrofuran or dioxane conveniently in the presence of a tertiary organic base such as triethylamine, pyridine or 2-dimethylaminopyridine, in the presence of N-ethyl-diisopropylamine (Hunig base), wherein these organic bases may simultaneously serve as solvent, or in the presence of an inorganic base such as sodium carbonate, potassium carbonate or sodium hydroxide solution expediently at temperatures between −50 and 150° C., preferably at temperatures between −20 and 80° C.

With a compound of general formula III wherein Z₁ denotes a hydroxy group, the reaction is preferably carried out in the presence of a dehydrating agent, e.g. in the presence of isobutyl chloroformate, thionylchloride, trimethylchlorosilane, phosphorus trichloride, phosphorus pentoxide, hexamethyldisilazane, N,N′-dicyclohexylcarbodiimide, O-(benzotriazol-1-yl)-N,N,N′,N′-tetramethyluronium tetrafluoroborate, N,N′-dicyclohexylcarbodiimide/N-hydroxysuccinimide or 1-hydroxy-benzotriazole and optionally additionally in the presence of 4-dimethylamino-pyridine, N,N′-carbonyldiimidazole or triphenylphosphine/carbon tetrachloride, expediently in a solvent such as methylene chloride, tetrahydrofuran, dioxane, toluene, chlorobenzene, dimethylsulphoxide, ethylene glycol diethyl ether or sulpholane and optionally in the presence of a reaction accelerator such as 4-dimethylaminopyridine at temperatures between −50 and 150° C., but preferably at temperatures between −20 and 80° C.

b. In order to prepare a compound of general formula I wherein R_{b} denotes a C₁₋₆-alkoxycarbonyl, C₃₋₇-cycloalkoxycarbonyl or phenyl-C₁₋₃-alkoxycarbonyl group or a R₃NR₄—CO group wherein R₃ and R₄ are as hereinbefore defined:

reacting a compound of general formula

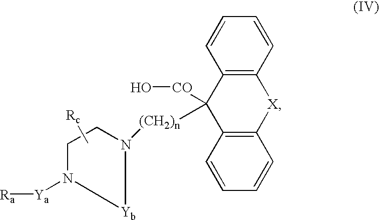

wherein

Rₐ, R_{c}, X, Yₐ, Y_{b} and n are as hereinbefore defined, with a compound of general formula

H—R_{b}′, (V)

wherein

R_{b}′ denotes a C₁₋₆-alkoxy, C₃₋₇-cycloalkoxy or phenyl-C₁₋₃-alkoxy group or a R₃NR₄ group, wherein R₃ and R₄ are as hereinbefore defined, or with the reactive derivatives thereof.

The reaction is expediently carried out with a corresponding halide or anhydride of general formula IV in a solvent such as methylene chloride, chloroform, carbon tetrachloride, ether, tetrahydrofuran, dioxane, benzene, toluene, acetonitrile or sulpholane optionally in the presence of an inorganic or organic base at temperatures between −20 and 200° C., but preferably at temperatures between −10 and 160° C. It may, however, also be carried out with the free acid optionally in the presence of an acid-activating agent or a dehydrating agent, e.g. in the presence of isobutyl chloroformate, thionylchloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide, TBTU or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

If according to the invention a compound of general formula I is obtained which contains an amino or alkylamino group it may be converted by acylation into a corresponding acyl compound, or

if a compound of general formula I is obtained which contains a nitro group, or may be converted by reduction into a corresponding amino compound.

The subsequent acylation is expediently carried out with a corresponding halide, anhydride or isocyanate in a solvent such as methylene chloride, chloroform, carbon tetrachloride, ether, tetrahydrofuran, dioxane, benzene, toluene, acetonitrile or sulpholane optionally in the presence of an inorganic or organic base at temperatures between −20 and 200° C., but preferably at temperatures between −10 and 160° C. However, it may also be carried out with the free acid optionally in the presence of an acid-activating agent or a dehydrating agent, e.g. in the presence of isobutyl chloroformate, thionylchloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide, TBTU or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

The subsequent reduction of a nitro group is expediently carried out hydrogenolytically, e.g. with hydrogen in the presence of a catalyst such as platinum, palladium/charcoal or Raney nickel in a suitable solvent such as methanol, ethanol, ethyl acetate, tetrahydrofuran, dioxane, dimethylformamide or glacial acetic acid, optionally with the addition of an acid such as hydrochloric acid and at a hydrogen pressure of 1 to 7 bar, but preferably 1 to 5 bar, with metals such as iron, tin or zinc in the presence of an acid such as acetic acid or hydrochloric acid, with salts such as iron(II)sulphate, tin (II) chloride, sodium sulphide, sodium hydrogen sulphite or sodium dithionite, or with hydrazine in the presence of Raney nickel at temperatures between 0 and 100° C., but preferably at temperatures between 20 and 60° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a trimethylsilyl, tert.butyl-dimethylsilyl, acetyl, benzoyl, methyl, ethyl, tert.butyl, trityl, benzyl or tetrahydropyranyl group,

a protecting group for a carboxyl group may be a trimethylsilyl, methyl, ethyl, tert.butyl, benzyl or tetrahydropyranyl group and

protecting groups for an amino, alkylamino or imino group may be a formyl, acetyl, trifluoroacetyl, ethoxycarbonyl, tert.butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C. However, a silyl group may also be cleaved using tetrabutylammonium fluoride as described hereinbefore.

However, a benzyl, methoxybenzyl or benzyloxycarbonyl group is cleaved for example hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid or by treating with iodotrimethylsilane, optionally using a solvent such as methylene chloride, dioxane, methanol or diethyl ether.

A trifluoroacetyl group is preferably cleaved by treating with an acid such as hydrochloric acid, optionally in the presence of a solvent such as acetic acid at temperatures between 50 and 120° C. or by treating with sodium hydroxide solution, optionally in the presence of a solvent such as tetrahydrofuran at temperatures between 0 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of geheral formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)-or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, phosphoric acid, fumaric acid, succinic acid, lactic acid, citric acid, tartaric acid or maleic acid.

Moreover, if the new compounds of formula I thus obtained contain an acidic group such as a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

The compounds of general formulae II to VI used as starting materials are known from the literature in some cases or may be obtained by methods known from the literature or are described in the Examples.

A compound of general formula II is obtained for example by reacting a compound of general formula

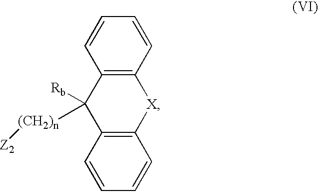

wherein

R_{b}, X and n are as hereinbefore defined and

Z₂ denotes a nucleofugic leaving group such as a chlorine or bromine atom, with a corresponding piperazine or homopiperazine wherein an imino group may conveniently be protected by a conventional protecting group, e.g. by a tert.butoxycarbonyl or benzyloxycarbonyl group, in a melt or in a solvent such as ethanol, dioxane, tetrahydrofuran, acetonitrile or dimethylformamide, in the presence of a base such as triethylamine or potassium carbonate and at temperatures between 0 and 130° C., but preferably at temperatures between 20 and 80° C. Any protecting group used is subsequently cleaved by methods known from the literature.

A compound of general formula IV is obtained for example analogously to method a) by reacting a correspondingly substituted carboxylic acid derivative with a compound of general formula III and optionally subsequently cleaving any protecting group used to protect the carboxy group.

As already mentioned hereinbefore, the compounds of general formula I and the physiologically acceptable salts thereof have valuable pharmacological properties. In particular, they are valuable inhibitors of the microsomal triglyceride-transfer protein (MTP) and are therefore suitable for lowering the plasma levels of the atherogenic lipoproteins.

For example, the compounds according to the invention were investigated for their biological effects as follows:

Inhibitors of MTP were identified by a cell-free MTP activity test. Solubilised liver microsomes from various species (e.g. rat, pig) can be used as the MTP source. To prepare the donor and acceptor vesicles, lipids dissolved in organic solvents were mixed in a suitable ratio and applied as a thin layer to the wall of a glass container by blowing the solvent in a nitrogen current. The solution used to prepare donor vesicles contained 400 μM of phosphatidyl choline, 75 μM of cardiolipin and 10 μM of [¹⁴C]-triolein (68,8 μCi/mg). To prepare the acceptor vesicles, a solution of 1.2 mM phosphatidyl choline, 5 μM triolein and 15 μM [³H]-dipalmitoylphosphatidyl choline (108 mCi/mg) was used. Vesicles are produced by wetting the dried lipids with test buffer and subsequently ultrasonicating. Vesicle populations of uniform size were obtained by gel filtration of the ultrasonicated lipids. The MTP activity test contains donor vesicles, acceptor vesicles as well as the MTP source in test buffer. Substances were added from concentrated DMSO-containing stock solutions, the final concentration of DMSO in the test was 0.1%. The reaction was started by the addition of MTP. After a corresponding incubation time the transfer process was stopped by the addition of 500 μl of a SOURCE 30Q anion exchanger suspension (Pharmacia Biotech). The mixture was shaken for 5 minutes and the donor vesicles bound to the anion exchanger material were separated off by centrifuging. The radioactivity of [³H] and [¹⁴C] in the supernatant was determined by liquid scintillation measurement and from this the recovery of the acceptor vesicles and the triglyceride transfer speed was calculated.

In view of the abovementioned biological properties the compounds of general formula I and the physiologically acceptable salts thereof are particularly suitable for lowering the plasma concentration of atherogenic apolipoprotein B (apoB)-containing lipoproteins such as chylomicrons and/or very low density lipoproteins (VLDL) as well as the residues thereof such as low density lipoproteins (LDL) and/or lipoprotein(a) (Lp(a)), for treating hyperlipidaemias, for preventing and treating atherosclerosis and the clinical sequelae thereof, and for preventing and treating related disorders such as diabetes mellitus, adiposity and pancreatitis, oral administration being preferred.

The daily dose needed to achieve such an effect is between 0.5 and 500 mg, expediently between 1 and 350 mg, but preferably between 5 and 200 mg, in adults.

For this purpose, the compounds of formula I prepared according to the invention, optionally combined with other active substances such as other lipid-lowering agents, for example HMG-CoA-reductase-inhibitors, cholesterol biosynthesis inhibitors such as squalene synthase inhibitors and squalene cyclase inhibitors, bile acid-binding resins, fibrates, cholesterol resorption inhibitors, niacin, probucol, CETP inhibitors and ACAT inhibitors may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat or suitable mixtures thereof into conventional galenic preparations such as plain or coated tablets, capsules, powders, suspensions or suppositories.

The Examples which follow are intended to illustrate the invention in more detail:

Preparation of the Starting Products:

### EXAMPLE I

9-(4-bromo-butyl)-9H-fluorene-9-carboxylic Acid

89 ml (0.11 mol) of a 1.6 molar n-butyl-lithium solution in hexane are added dropwise at 0° C. to a solution of 21 g (0.1 mol) of 9-fluorenecarboxylic acid in 700 ml tetrahydrofuran under nitrogen and the mixture is stirred for one hour. Then, again at 0° C., 13.13 ml (0.11 mol) of dibromobutane are added and the solution is stirred for 30 hours at ambient temperature. After this time 50 ml of water are added and the mixture is stirred for 30 minutes. The solution is concentrated by evaporation, mixed with water and extracted with 250 ml of diethylether. The aqueous phase is acidified with 150 ml of 1N hydrochloric acid and extracted three times with 250 ml of dichloromethane. The combined organic phases are dried over sodium sulphate and the solvent is eliminated.

Yield: 18.5 g (53.6% of theory),

melting point: 123° C.

The following compounds are prepared analogously to Example I:

(1) 9-(4-bromo-butyl)-9H-xanthene-9-carboxylic acid Prepared from xanthene-9-carboxylic acid and dibromobutane

(2) methyl (3-bromo-propyl)-9H-fluorene-9-carboxylate Prepared from methyl fluorene-9-carboxylate and dibromopropane

### EXAMPLE II

9-(4-bromo-butyl)-9H-fluorene-9-carboxylic Acid Chloride

23 g (0.067 mol) of 9-(4-bromo-butyl)-9H-fluorene-9-carboxylic acid are dissolved in 40 ml of dichloromethane, and combined with three drops of dimethylformamide and 6.96 ml (0.081 mol) of oxalyl chloride, dissolved in 10 ml of dichloromethane, under nitrogen at 0° C. The mixture is stirred for 3 hours at ambient temperature. Then the solvent is eliminated and the crude product is further reacted without further purification.

Yield: 24 g (99% of theory)

The following compounds are prepared analogously to Example II:

(1) 9-(4-bromo-butyl)-9H-xanthene-9-carboxylic acid chloride

(2) 9-[3-(4-phenylacetyl-piperazino)-propyl]-9H-fluorene-9-carboxylic acid chloride

Prepared from 9-[3-(4-phenylacetyl-piperazino)-propyl]-9H-fluorene-9-carboxylic acid

### EXAMPLE III

9-(4-bromo-butyl)-9H-fluorene-9-carboxylic acid-(2,2,2-trifluoro-ethyl)-amide

23 g (0.063 mol) of 9-(4-bromo-butyl)-9H-fluorene-9-carboxylic acid chloride are added dropwise to a solution of 9.35 g (0.069 mol) of 2,2,2-trifluoroethylamine-hydrochloride and 26 ml (0.188 mol) of triethylamine in 550 ml of dichloromethane at 0° C. under nitrogen and stirred for 2 hours at ambient temperature. The reaction mixture is extracted twice with water, 1N hydrochloric acid and sodium hydrogen carbonate solution. The organic phase is dried over sodium sulphate and the solvent is distilled off. The product is purified by column chromatography on silica gel (eluant: cyclohexane/ethyl acetate=8:1).

Yield: 15.8 g (58.6% of theory),

melting point: 172° C.

The following compounds are prepared analogously to Example III:

(1) 9-(4-bromo-butyl)-9H-xanthene-9-carboxylic acid-(2,2,2-trifluoro-ethyl)-amide

(2) 9-(4-bromo-butyl)-9H-fluorene-9-carboxylic acid-propylamide

(3) 9-(4-bromo-butyl)-9H-fluorene-9-carboxylic acid-benzylamide

(4) 9-(4-bromo-butyl)-9H-fluorene-9-carboxylic acid-phenylamide

(5) 9-(4-bromo-butyl)-9H-fluorene-9-carboxylic acid-cyclopentylamide

(6) 9-(4-bromo-butyl)-9H-fluorene-9-carboxylic acid-N-methyl-N-ethylamide

### EXAMPLE IV

9-[4-(4-tert.butyloxycarbonyl-piperazino)-butyl]-9H-fluorene-9-carboxylic acid-(2,2,2-trifluoroethyl)amide

A solution of 1.6 g (8.59 mmol) of tert. butyl piperazine-1-carboxylate, 3.7 g (8.68 mmol) of 9-(4-bromo-butyl)-9H-fluorene-9-carboxylic acid-(2,2,2-trifluoro-ethyl)-amide and 2.6 g (20.15 mmol) of ethyldiisopropylamine in 80 ml of DMF is stirred for 40 hours at 40° C. The DMF is distilled off using the rotary evaporator. The residue is taken up in dichloromethane and extracted with an ammonium hydroxide solution. The organic phase is dried over sodium sulphate and the solvent is distilled off. The product is purified by column chromatography on silica gel (eluant: dichloromethane/ethanol=19:1).

Yield: 4.6 g (99.7% of theory),

C₂₉H₃₆F₃N₃O₃ (M=531.62)

Calc.: molecular peak (M+H)⁺: 532

Found: molecular peak (M+H)⁺: 532

The following compounds are prepared analogously to Example IV:

(1) 9-[4-(4-tert.butyloxycarbonyl-piperazino)-butyl]-9H-xanthene-9-carboxylic acid-(2,2,2-trifluoroethyl)amide

(2) 9-[4-(4-tert.butyloxycarbonyl-(S)-2-methyl-piperazino)-butyl]-9H-fluorene-9-carboxylic acid-(2,2,2-trifluoroethyl)amide

(3) 9-[4-(4-tert.butyloxycarbonyl-(R)-2-methyl-piperazino)-butyl]-9H-fluorene-9-carboxylic acid-(2,2,2-trifluoroethyl)amide

(4) 9-[4-(4-tert.butyloxycarbonyl-[1,4]diazepan-1-yl)-butyl]-9H-fluorene-9-carboxylic acid-(2,2,2-trifluoroethyl)amide

(5) methyl 9-[3-(4-tert.butyloxycarbonyl-piperazino)propyl]-9H-fluorene-9-carboxylate

### EXAMPLE V

9-(4-piperazino-butyl)-9H-fluorene-9-carboxylic acid-(2,2,2-trifluoro-ethyl)-amide

A solution of 4.6 g (8.65 mmol) of 9-[4-(4-tert.butyloxy-carbonyl-piperazino)-butyl]-9H-fluorene-9-carboxylic acid-(2,2,2-trifluoro-ethyl)-amide and 20 ml of trifluoroacetic acid in 200 ml of dichloromethane is stirred for two hours at ambient temperature. Then the reaction solution is concentrated by evaporation using the rotary evaporator, the residue is taken up in dichloromethane and extracted with an ammonium hydroxide solution. The organic phase is dried over sodium sulphate and the solvent is distilled off. The product is purified by column chromatography on silica gel (eluant: dichloromethane/ethanol=9:1).

Yield: 3.6 g (96,4% of theory),

C₂₄H₂₈F₃N₃O (M=431.50)

Calc.: molecular peak (M+H)⁺: 432

Found: molecular peak (M+H)⁺: 432

The following compounds are prepared analogously to Example V:

(1) 9-(4-piperazino-butyl)-9H-xanthene-9-carboxylic acid-(2,2,2-trifluoro-ethyl)-amide

(2) 9-[4-((S)-2-methyl-piperazino)-butyl]-9H-fluorene-9-carboxylic acid-(2,2,2-trifluoroethyl)amide

(3) 9-[4-((R)-2-methyl-piperazino)-butyl]-9H-fluorene-9-carboxylic acid-(2,2,2-trifluoroethyl)amide

(4) 9-[4-([1,4]diazepan-1-yl)-butyl]-9H-fluorene-9-carboxylic acid-(2,2,2-trifluoroethyl)amide

(5) methyl 9-(3-piperazino-propyl)-9H-fluorene-9-carboxylate

Yield: 98% of theory,

C₂₂H₂₆N₂O₂ (M=350.46)

Calc.: molecular peak (M+H)⁺: 351

Found: molecular peak (M+H)⁺: 351

### EXAMPLE VI

9-[3-(4-phenylacetyl-piperazino)-propyl]-9H-fluorene-9-carboxylic Acid

3.5 g of methyl 9-[3-(4-phenylacetyl-piperazino)-propyl]-9H-fluorene-9-carboxylate (Example 12) are taken up in 80 ml of methanol/dioxane (1:1) and stirred for 2 hours with 38 ml of 1N sodium hydroxide solution at 50° C. Then it is acidified and extracted with methylene chloride. The organic phase is concentrated by rotary evaporation.

Yield: 2.7 g

### EXAMPLE VII

Methyl 9-(4-{4-[phenyl-acetyl]-piperazin-2-on-1-yl}-butyl)-9H-fluorene-9-carboxylate

1.4 g of 4-phenylacetyl-piperazin-2-one (prepared from piperazine-2-one and phenylacetic acid chloride) are dissolved in 30 ml of dimethylformamide and stirred with 0.3 g of sodium hydride at ambient temperature for 1.5 h. Then 2.3 g of methyl 9-(4-bromo-butyl)-9H-fluorene-9-carboxylate are added batch-wise and stirred for a further 4 h at ambient temperature. Then the solvent is evaporated off, the residue is taken up in methylene chloride and washed with water. The organic phase is concentrated by rotary evaporation and the residue is chromatographed through a silica gel column with methylene chloride/ethanol 19:1.

Yield: 87% of theory

Preparation of the End Products:

### EXAMPLE 1

9-[4-(4-phenylacetyl-piperazino)-butyl]-9H-fluorene-9-carboxylic acid-(2,2,2-trifluoro-ethyl)-amide

0.15 g of (1.5 mmol) of triethylamine and 0.11 g (0.712 mmol) of phenylacetic acid chloride, dissolved in 5 ml of dichloromethane, are successively added dropwise to a solution of 0.3 g (0.695 mmol) of 9-(4-piperazino-butyl)-9H-fluorene-9-carboxylic acid-(2,2,2-trifluoro-ethyl)-amide in 20 ml of dichloromethane and stirred for one hour at ambient temperature. The reaction mixture is extracted with an ammonium hydroxide solution. The organic phase is dried over sodium sulphate and the solvent is distilled off. The product is purified by column chromatography on silica gel (eluant: dichloromethane and then dichloromethane/ethanol=19:1).

Yield: 0.25 g (65.4% of theory),

C₃₂H₃₄F₃N₃O₂ (M=549.64)

Calc.: molecular peak (M+H)⁺: 550

Found: molecular peak (M+H)⁺: 550

### EXAMPLE 2

9-[4-(4-cyclohexylacetyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and cyclohexylacetyl chloride.

Yield: 0.35 g (90.6% of theory),

C₃₂H₄0F₃N₃O₂ (M=555.69)

Calc.: molecular peak (M+H)⁺: 556

Found: molecular peak (M+H)⁺: 556

### EXAMPLE 3

9-[4-(4-Propionyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and propionic acid chloride.

Yield: 0.3 g (88.5% of theory),

C₂₇H₃₂F₃N₃O₂ (M=487.57)

Calc.: molecular peak (M+H)⁺: 488

Found: molecular peak (M+H)⁺: 488

### EXAMPLE 4

9-[4-(4-benzoyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and benzoyl chloride.

Yield: 0.23 g (92.7% of theory),

C₃₁H₃₂F₃N₃O₂ (M=535.61)

Calc.: molecular peak (M+H)⁺: 536

Found: molecular peak (M+H)⁺: 536

### EXAMPLE 5

9-{4-[4-(4-phenyl-butyryl)-piperazino]-butyl}-9H-fluorene 9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and 4-phenylbutyric acid chloride.

Yield: 0.26 g (97.2% of theory),

C₃₄H₃₈F₃N₃O₂ (M=577.69)

Calc.: molecular peak (M+H)⁺: 578

Found: molecular peak (M+H)⁺: 578

### EXAMPLE 6

9-{4-[4-(2,5-dimethoxy-phenylacetyl)-piperazino]-butyl}-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and 2,5-dimethoxy-phenylacetic acid chloride.

Yield: 0.26 g (92.1% of theory),

C₃₄H₃₈F₃N₃O₄ (M=609.69)

Calc.: molecular peak (M+H)⁺: 610

Found: molecular peak (M+H)⁺: 610

### EXAMPLE 7

9-{4-[4-(3.4-dimethoxy-phenylacetyl)-piperazino]-butyl}-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and 3.4-dimethoxy-phenylacetic acid chloride.

Yield: 0.22 g (77.9% of theory),

C₃₄H₃₈F₃N₃O₄ (M=609.69)

Calc.: molecular peak (M+H)⁺: 610

Found: molecular peak (M+H)⁺: 610

### EXAMPLE 8

9-[4-(4-benzylsulphonyl-piperazino-butyl)-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and benzylsulphonic acid chloride.

Yield: (49% of theory),

C₃₁H₂₄F₃N₃O₃S (M=585.69)

Calc.: molecular peak (M+H)⁺: 586

Found: molecular peak (M+H)⁺: 586

### EXAMPLE 9

9-[4-(4-toluenesulphonyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and toluenesulphonic acid chloride.

Yield: (81% of theory),

C₃₁H₂₄F₃N₃O₃S (M=585.69)

Calc.: molecular peak (M+H)⁺: 586

Found: molecular peak (M+H)⁺: 586

### EXAMPLE 10

9-[4-(4-phenylacetyl-piperazino)-butyl]-9H-xanthene-9-carboxylic acid-(2,2,2-trifluoro-ethyl)-amide

Prepared analogously to Example 1 from 9-(4-piperazino-butyl)-9H-xanthene-9-carboxylic acid-(2,2,2-trifluoro-ethyl)-amide and phenylacetic acid chloride.

Yield: 0.4 g (91% of theory),

C₃₂H₃₄F₃N₃O₃ (M=565.64)

Calc.: molecular peak (M−H)⁺: 564

Found: molecular peak (M−H)⁺: 564

### EXAMPLE 11

9-[4-(4-chlorophenylacetyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and 4-chlorophenylacetic acid chloride.

Yield: 0.3 g (69% of theory),

C₃₂H₃₃ClF₃N₃O₂ (M=584.09)

Calc.: molecular peak (M−H)⁺: 582/584

Found: molecular peak (M−H)⁺: 582/584

### EXAMPLE 12

Methyl 9-[3-(4-phenylacetyl-piperazino)-propyl]-9H-fluorene-9-carboxylate

Prepared analogously to Example 1 from methyl 9-(3-piperazino)-propyl]-9H-fluorene-9-carboxylate and phenylacetic acid chloride.

Yield: 3.6 g (53% of theory),

C₃₀H₃₂N₂O₃ (M=468.60)

Calc.: molecular peak (M−H)⁺: 469

Found: molecular peak (M−H)⁺: 469

### EXAMPLE 13

9-[4-(4-phenoxyacetyl-piperazino)-butyl]-9H-fluorene-9-carboxylic acid-(2,2,2-trifluoro-ethyl)-amide

Prepared analogously to Example 1 from 9-(4-piperazino-butyl)-9H-fluorene-9-carboxylic acid-(2,2,2-trifluoro-ethyl)-amide and phenoxyacetic acid chloride.

Yield: 0.3 g (89% of theory),

C₃₂H₃₄F₃N₃O₃ (M=565.64)

Calc.: molecular peak (M+H)⁺: 566

Found: molecular peak (M+H)⁺: 566

The following compounds are prepared analogously to Example 13:

(1) 9-(4-{4-[(4-nitrophenyl)-acetyl]-piperazino}-butyl)-9H-fluorene-9-carboxylic acid-(2,2,2-trifluoro-ethyl)-amide

Yield: 57% of theory,

C₃₂H₃₃F₃N₄O₄ (M=594.63)

Calc.: molecular peak (M+H)⁺: 595

Found: molecular peak (M+H)⁺: 595

(2) 9-(4-{4-[2,2-diphenyl-acetyl]-piperazino}-butyl)-9H-fluorene-9-carboxylic acid-(2,2,2-trifluoro-ethyl)-amide

Yield: 80% of theory,

C₃₈H₃₈F₃N₃O₂ (M=625.74)

Calc.: molecular peak (M+H)⁺: 626

Found: molecular peak (M+H)⁺: 626

(3) 9-(4-{4-[(4-fluorophenyl)-acetyl]-piperazino}-butyl)-9H-fluorene-9-carboxylic acid-(2,2,2-trifluoro-ethyl)-amide

Yield: 63% of theory,

C₃₂H₃₃F₄N₃O₂ (M=567.63)

Calc.: molecular peak (M+H)⁺: 568

Found: molecular peak (M+H)⁺: 568

(4) 9-(4-{4-[2-phenyl-butyryl]-piperazino}-butyl)-9H-fluorene-9-carboxylic acid-(2,2,2-trifluoro-ethyl)-amide

Yield: 97% of theory,

C₁₄H₃₈F₃N₃O₂ (M=577.69)

Calc.: molecular peak (M+H)⁺: 578

Found: molecular peak (M+H)⁺: 578

(5) 9-(4-{4-[2-phenyl-2-acetoxy-acetyl]-piperazino}-butyl)-9H-fluorene-9-carboxylic acid-(2,2,2-trifluoro-ethyl)-amide

Yield: 94% of theory,

C₃₄H₃₆F₃N₃O₄ (M=607.67)

Calc.: molecular peak (M+H)⁺: 608

Found: molecular peak (M+H)⁺: 608

(6) 9-(4-{4-[phenyl-acetyl]-(S)-2-methyl-piperazino}-butyl)-9H-fluorene-9-carboxylic acid-(2,2,2-trifluoro-ethyl)-amide

Yield: 79% of theory,

C₃₃H₃₆F₃N₃O₂ (M=563.66)

Calc.: molecular peak (M+H)⁺: 564

Found: molecular peak (M+H)⁺: 564

(7) 9-(4-{4-[phenyl-acetyl]-(R)-2-methyl-piperazino}-butyl)-9H-fluorene-9-carboxylic acid-(2,2,2-trifluoro-ethyl)-amide

Yield: 68% of theory,

C₃₃H₃₆F₃N₃O₂ (M=563.66)

Calc.: molecular peak (M+H)⁺: 564

Found: molecular peak (M+H)⁺: 564

(8) 9-(4-{4-(benzyloxycarbonyl-piperazino}-butyl)-9H-fluorene-9-carboxylic acid-(2,2,2-trifluoro-ethyl)-amide

Yield: 63% of theory,

C₃₂H₃₄F₃N₃O₃ (M=565.64)

Calc.: molecular peak (M+H)⁺: 566

Found: molecular peak (M+H)⁺: 566

(9) 9-(4-{4-(3-phenylpropionyl)-piperazino)}-butyl)-9H-fluorene-9-carboxylic acid-(2,2,2-trifluoro-ethyl)-amide

Yield: 84% of theory,

C₃₃H₃₆F₃N₃O₂ (M=563.66)

Calc.: molecular peak (M+H)⁺: 564

Found: molecular peak (M+H)⁺: 564

(10) 9-(4-{4-hexanoyl-piperazino}-butyl)-9H-fluorene-9-carboxylic acid-(2,2,2-trifluoro-ethyl)-amide

Yield: 95% of theory,

C₃₀H₃₈F₃N₃O₂ (M=529.65)

Calc.: molecular peak (M+H)⁺: 530

Found: molecular peak (M+H)⁺: 530

(11) 9-(4-{4-(2-bromo-benzoyl)-piperazino)}-butyl)-9H-fluorene-9-carboxylic acid-(2,2,2-trifluoro-ethyl)-amide

Yield: 89% of theory,

C₃₁H₃₁BrF₃N₃O₂ (M=614.51)

Calc.: molecular peak (M+H)⁺: 614/616

Found: molecular peak (M+H)⁺: 614/616

(12) 9-(4-{4-(3-bromo-benzoyl)-piperazino)}-butyl)-9H-fluorene-9-carboxylic acid-(2,2,2-trifluoro-ethyl)-amide

Yield: 88% of theory,

C₃₁H₃₁BrF₃N₃O₂ (M=614.51)

Calc.: molecular peak (M+H)⁺: 614/616

Found: molecular peak (M+H)⁺: 614/616

(13) 9-(4-{4-[N-methyl-N-phenylcarbamoyl]-piperazino}-butyl)-9H-fluorene-9-carboxylic acid-(2,2,2-trifluoro-ethyl)-amide (Prepared from N-methyl-N-phenyl-carbamoyl-chloride)

Yield: 96% of theory,

C₃₂H₃₅F₃N₄O₂ (M=564.65)

Calc.: molecular peak (M+H)⁺: 565

Found: molecular peak (M+H)⁺: 565

(14) 9-(4-{4-(phenyl-acetyl)-[1.4]diazepan-1-yl}-butyl)-9H-fluorene-9-carboxylic acid-(2,2,2-trifluoro-ethyl)-amide

Yield: 52% of theory,

C₃₃H₃₆F₃N₃O₂ (M=563.66)

Calc.: molecular peak (M+H)⁺: 564

Found: molecular peak (M+H)⁺: 564

### EXAMPLE 14

9-{4-[4-(4-trifluoromethyl-phenylacetyl)-piperazino]-butyl}-9H-fluorene-9-carboxylic acid-(2,2,2-trifluoro-ethyl)-amide

A solution of 0.3 g (0.695 mmol) of 9-(4-piperazino-butyl)-9H-fluorene-9-carboxylic acid-(2,2,2-trifluoro-ethyl)-amide, 0.14 g (0.686 mmol) of 4-(trifluoromethyl)-phenylacetic acid, 1.27 g (9.845 mmol) of N-ethyl-diisopropylamine and 0.45 g (1.402 mmol) of TBTU in 10 ml of dimethylformamide is stirred for 20 hours at ambient temperature. Then the reaction solution is concentrated by evaporation using the rotary evaporator, the residue is taken up in dichloromethane and extracted with an ammonium hydroxide solution. The organic phase is dried over sodium sulphate and the solvent is distilled off. The product is purified by column chromatography through silica gel (dichloromethane, then dichloromethane/ethanol=19:1).

Yield: 0.3 g (69.9% of theory),

C₃₃H₃₃F₆N₃O₂ (M=617.64)

Calc.: molecular peak (M+H)⁺: 618

Found: molecular peak (M+H)⁺: 618

The following compounds are prepared analogously to Example 14:

(1) 9-(4-{4-[(3-chlorophenyl)-acetyl]-piperazino}-butyl)-9H-fluorene-9-carboxylic acid-(2,2,2-trifluoro-ethyl)-amide

Yield: 49% of theory,

C₃₂H₃₃ClF₃N₃O₂ (M=584.08)

Calc.: molecular peak (M+H)⁺: 584/586

Found: molecular peak (M+H)⁺: 584/586

(2) 9-(4-{4-[(3-trifluoromethylphenyl)-acetyl]-piperazino}-butyl)-9H-fluorene-9-carboxylic acid-(2,2,2-trifluoro-ethyl)-amide

Yield: 65% of theory,

C₃₃H₃₃F₆N₃O₂ (M=617.64)

Calc.: molecular peak (M+H)⁺: 618

Found: molecular peak (M+H)⁺: 618

(3) 9-(4-{4-[(4-cyanophenyl)-acetyl]-piperazino}-butyl)-9H-fluorene-9-carboxylic acid-(2,2,2-trifluoro-ethyl)-amide

Yield: 62% of theory,

C₃₃H₃₃F₃N₄O₂ (M=574.65)

Calc.: molecular peak (M+H)⁺: 575

Found: molecular peak (M+H)⁺: 575

(4) 9-(4-{4-[(4-methoxymethyl-phenyl)-acetyl]-piperazino}-butyl)-9H-fluorene-9-carboxylic acid-(2,2,2-trifluoro-ethyl)-amide

Yield: 72% of theory,

C₃₄H₃₈F₃N₃O₃ (M=593.69)

Calc.: molecular peak (M+H)⁺: 594

Found: molecular peak (M+H)⁺: 594

(5) 9-(4-{4-[(2,6-dichlorophenyl)-acetyl]-piperazino)-butyl)-9H-fluorene-9-carboxylic acid-(2,2,2-trifluoro-ethyl)-amide

Yield: 81% of theory,

C₃₂H₃₂Cl₂F₃N₃O₂ (M=618.53)

Calc.: molecular peak (M+H)⁺: 616/618/620

Found: molecular peak (M+H)⁺: 616/618/620

(6) 9-(4-{4-[(2,4-dichlorophenyl)-acetyl]-piperazino}-butyl)-9H-fluorene-9-carboxylic acid-(2,2,2-trifluoro-ethyl)-amide

Yield: 81% of theory,

C₃₂H₃₂Cl₂F₃N₃O₂ (M=618.53)

Calc.: molecular peak (M+Na)⁺: 640/642/644

Found: molecular peak (M+Na)⁺: 640/642/644

(7) 9-(4-{4-[(2,3-difluorophenyl)-acetyl]-piperazino}-butyl)-9H-fluorene-9-carboxylic acid-(2,2,2-trifluoro-ethyl)-amide

Yield: 68% of theory,

C₃₂H₃₂F₅N₃O₂ (M=585.62)

Calc.: molecular peak (M+H)⁺: 586

Found: molecular peak (M+H)⁺: 586

(8) 9-(4-{4-[(2,3,6-trichlorophenyl)-acetyl]-piperazino}-butyl)-9H-fluorene-9-carboxylic acid-(2,2,2-trifluoro-ethyl)-amide

Yield: 77% of theory,

C₃₂H₃₁Cl₃F₃N₃O₂ (M=652.97)

Calc.: molecular peak (M+H)⁺: 652/654/656

Found: molecular peak (M+H)⁺: 652/654/656

(9) 9-(4-{4-[(3-bromophenyl)-acetyl]-piperazino}-butyl)-9H-fluorene-9-carboxylic acid-(2,2,2-trifluoro-ethyl)-amide

Yield: 98% of theory,

C₃₂H₃₃BrF₃N₃O₂ (M=628.53)

Calc.: molecular peak (M−H)⁻: 626/628

Found: molecular peak (M−H)⁻: 626/628

(10) 9-(4-{4-[(3-fluorophenyl)-acetyl]-piperazino}-butyl)-9H-fluorene-9-carboxylic acid-(2,2,2-trifluoro-ethyl)-amide

Yield: 98% of theory,

C₃₂H₃₃F₄N₃O₂ (M=567.63)

Calc.: molecular peak (M−H)⁻: 566

Found: molecular peak (M−H)⁻: 566

(11) 9-(4-{4-[(3,5-difluorophenyl)-acetyl]-piperazino}-butyl)-9H-fluorene-9-carboxylic acid-(2,2,2-trifluoro-ethyl)-amide

Yield: 77% of theory,

C₃₂H₃₂F₅N₃O₂ (M=585.62)

Calc.: molecular peak (M+H)⁺: 586

Found: molecular peak (M+H)⁺: 586

(12) 9-(4-{4-[(2,5-difluorophenyl)-acetyl]-piperazino}-butyl)-9H-fluorene-9-carboxylic acid-(2,2,2-trifluoro-ethyl)-amide

Yield: 98% of theory,

C₃₂H₃₂F₅N₃O₂ (M=585.62)

Calc.: molecular peak (M+H)⁺: 586

Found: molecular peak (M+H)⁺: 586

(13) 9-(4-{4-[(2-hydroxyphenyl)-acetyl]-piperazino}-butyl)-9H-fluorene-9-carboxylic acid-(2,2,2-trifluoro-ethyl)-amide

Yield: 38% of theory,

C₃₂H₃₄F₃N₃O₃ (M=565.64)

Calc.: molecular peak (M+H)⁺: 566

Found: molecular peak (M+H)⁺: 566

(14) 9-(4-{4-[(3,4-dihydroxyphenyl)-acetyl]-piperazino}-butyl)-9H-fluorene-9-carboxylic acid-(2,2,2-trifluoro-ethyl)-amide

Yield: 44% of theory,

C₃₂H₃₄F₃N₃O₄ (M=581.64)

Calc.: molecular peak (M+H)⁺: 582

Found: molecular peak (M+H)⁺: 582

(15) 9-(4-{4-[(3,4-methylenedioxy-phenyl)-acetyl]-piperazino}-butyl)-9H-fluorene-9-carboxylic acid-(2,2,2-trifluoro-ethyl)-amide

Yield: 86% of theory,

C₃₃H₃₄F₃N₃O₄ (M=593.65)

Calc.: molecular peak (M+H)⁺: 594

Found: molecular peak (M+H)⁺: 594

(16) 9-(4-{4-[(3,4-dichlorophenyl)-acetyl]-piperazino}-butyl)-9H-fluorene-9-carboxylic acid-(2,2,2-trifluoro-ethyl)-amide

Yield: 88%: of theory,

C₃₂H₃₂Cl₂F₃N₃O₂ (M=618.53)

Calc.: molecular peak (M+H)⁺: 619

Found: molecular peak (M+H)⁺: 619

(17) 9-(4-{4-[(4-methylphenyl)-acetyl]-piperazino}-butyl)-9H-fluorene-9-carboxylic acid-(2,2,2-trifluoro-ethyl)-amide

Yield: 63% of theory,

C₃₃H₃₆F₃N₃O₂ (M=563.66)

Calc.: molecular peak (M+H)⁺: 564

Found: molecular peak (M+H)⁺: 564

(18) 9-(4-{4-[2-(2,3,4,5,6-pentafluorophenyl)-acetyl]-piperazino}-butyl)-9H-fluorene-9-carboxylic acid-(2,2,2-trifluoro-ethyl)-amide

Yield: 87% of theory,

C₃₂H₂₉F₈N₃O₂ (M=639.59)

Calc.: molecular peak (M−H)⁻: 638

Found: molecular peak (M−H)⁻: 638

(19) 9-(4-{4-[2-(2L)-hydroxy-2-phenyl-acetyl]-piperazino}-butyl)-9H-fluorene-9-carboxylic acid-(2,2,2-trifluoro-ethyl)-amide

Yield: 63% of theory,

C₃₂H₃₄F₃N₃O₃ (M=565.64)

Calc.: molecular peak (M+H)⁺: 566

Found: molecular peak (M+H)⁺: 566

(20) 9-(4-{4-[2-(2D)-hydroxy-2-phenyl-acetyl]-piperazino}-butyl)-9H-fluorene-<9-carboxylic acid-(2,2,2-trifluoro-ethyl)-amide

Yield: 34% of theory,

C₃₂H₃₄F₃N₃O₃ (M=565.64)

Calc.: molecular peak (M+H)⁺: 566

Found: molecular peak (M+H)⁺: 566

(21) 9-(4-{4-[2-oxo-2-phenyl-acetyl]-piperazino}-butyl)-9H-fluorene-9-carboxylic acid-(2,2,2-trifluoro-ethyl)-amide

Yield: 46% of theory,

C₃₂H₃₂F₃N₃O₃ (M=563.62)

Calc.: molecular peak (M+H)⁺: 564

Found: molecular peak (M+H)⁺: 564

(22) 9-(4-{4-[2-oxo-2-(3-chlorophenyl)-acetyl]-piperazino}-butyl)-9H-fluorene-9-carboxylic acid-(2,2,2-trifluoro-ethyl)-amide

Yield: 65% of theory,

C₃₂H₃₁ClF₃N₃O₃ (M=598.07)

Calc.: molecular peak (M−H)⁻: 596/598

Found: molecular peak (M−H)⁻: 596/598

(23) 9-(4-{4-[(2,4-dichlorophenyl)-acetyl]-(S)-2-methyl-piperazino}-butyl)-9H-fluorene-9-carboxylic acid-(2,2,2-trifluoro-ethyl)-amide

Yield: 47% of theory,

C₃₃H₃₄Cl₂F₃N₃O₂ (M=632.55)

Calc.: molecular peak (M+H)⁺: 632/634/636

Found: molecular peak (M+H)⁺: 632/634/636

(24) 9-(4-{4-[(2,4-dichlorophenyl)-acetyl]-(R)-2-methyl-piperazino}-butyl)-9H-fluorene-9-carboxylic acid-(2,2,2-trifluoro-ethyl)-amide

Yield: 11% of theory,

C₃₃H₃₄Cl₂F₃N₃O₂ (M=632.55)

Calc.: molecular peak (M+H)⁺: 632/634/636

Found: molecular peak (M+H)⁺: 632/634/636

(25) 9-(4-{4-[2-oxo-2-phenyl-acetyl]-(S)-2-methyl-piperazino}-butyl)-9H-fluorene-9-carboxylic acid-(2,2,2-trifluoro-ethyl)-amide

Yield: 72% of theory,

C₃₃H₃₄F₃N₃O₃ (M=577.65)

Calc.: molecular peak (M+H)⁺: 578

Found: molecular peak (M+H)⁺: 578

(26) 9-(4-{4-[2-oxo-2-phenyl-acetyl]-(R)-2-methyl-piperazino}-butyl)-9H-fluorene-9-carboxylic acid-(2,2,2-trifluoro-ethyl)-amide

Yield: 43% of theory,

C₃₃H₃₄F₃N₃O₃ (M=577.65)

Calc.: molecular peak (M+H)⁺: 578

Found: molecular peak (M+H)⁺: 578

(27) 9-(4-{4-(1,2,3,4-tetrahydro-naphthalene-2-carbonyl)-piperazino}-butyl)-9H-fluorene-9-carboxylic acid-(2,2,2-trifluoro-ethyl)-amide

Yield: 97% of theory, C₃₅H₃₈F₃N₃O₂ (M=589.70)

Calc.: molecular peak (M+H)⁺: 590

Found: molecular peak (M+H)⁺: 590

(28) 9-(4-{4-(4-trifluoromethyl-benzoyl)-piperazino}-butyl)-9H-fluorene-9-carboxylic acid-(2,2,2-trifluoro-ethyl)-amide

Yield: 90% of theory,

C₃₂H₃₁F₆N₃O₂ (M=603.61)

Calc.: molecular peak (M+H)⁺: 604

Found: molecular peak (M+H)⁺: 604

(29) 9-(4-{4-(4-(pyridin-2-yl-acetyl)-piperazino}-butyl)-9H-fluorene-9-carboxylic acid-(2,2,2-trifluoro-ethyl)-amide

Yield: 78% of theory,

C₃₁H₃₃F₃N₄O₂ (M=550.62)

Calc.: molecular peak (M+H)⁺: 551

Found: molecular peak (M+H)⁺: 551

(30) 9-(4-{4-(4-(pyridin-3-yl-acetyl)-piperazino}-butyl)-9H-fluorene-9-carboxylic acid-(2,2,2-trifluoro-ethyl)-amide

Yield: 77% of theory,

C₃₁H₃₃F₃N₄O₂ (M=550.62)

Calc.: molecular peak (M+H)⁺: 551

Found: molecular peak (M+H)⁺: 551

(31) 9-(4-{4-(4-(2-1H-indol-3-yl-acetyl)-piperazino}-butyl)-9H-fluorene-9-carboxylic acid-(2,2,2-trifluoro-ethyl)-amide

Yield: 61% of theory,

C₃₄H₃₅F₃N₄O₂ (M=588.67)

Calc.: molecular peak (M+H)⁺: 589

Found: molecular peak (M+H)⁺: 589

(32) 9-(4-{4-[(3-methylphenyl)-acetyl]-piperazino}-butyl)-9H-fluorene-9-carboxylic acid-(2,2,2-trifluoro-ethyl)-amide

Yield: 71% of theory,

C₃₃H₃₆F₃N₃O₂ (M=563.66)

Calc.: molecular peak (M+H)⁺: 564

Found: molecular peak (M+H)⁺: 564

(33) 9-(4-{4-[3-(3-cyanophenyl)-propionyl]-piperazino}-butyl)-9H-fluorene-9-carboxylic acid-(2,2,2-trifluoro-ethyl)-amide

Yield: 73% of theory,

C₃₄H₃₅F₃N₄O₂ (M=588.67)

Calc.: molecular peak (M+H)⁺: 589

Found: molecular peak (M+H)⁺: 589

(34) 9-(4-{4-[3-(4-cyanophenyl)-propionyl]-piperazino}-butyl)-9H-fluorene-9-carboxylic acid-(2,2,2-trifluoro-ethyl)-amide

Yield: 68% of theory,

C₃₄H₃₅F₃N₄O₂ (M=588.67)

Calc.: molecular peak (M+H)⁺: 589

Found: molecular peak (M+H)⁺: 589

(35) 9-(4-{4-[(fluoren-9-yl)-acetyl]-piperazino)-butyl)-9H-fluorene-9-carboxylic acid-(2,2,2-trifluoro-ethyl)-amide

Yield: 60% of theory,

C₃₉H₃₈F₃N₃O₂ (M=637.75)

Calc.: molecular peak (M+H)⁺: 638

Found: molecular peak (M+H)⁺: 638

### EXAMPLE 15

9-{4-[4-(4-bromo-phenylacetyl)-piperazino]-butyl})-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and 4-bromo-phenylacetic acid.

Yield: 0.15 g (34.3% of theory),

C₃₂H₃₃BrF₃N₃O₂ (M=628.53)

Calc.: molecular peak (M+H)⁺: 628/630

Found: molecular peak (M+H)⁺: 628/630

### EXAMPLE 16

9-{4-[4-(3-cyclohexyl-propionyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and 3-cyclohexyl-propionic acid.

Yield: 0.2 g (50.5% of theory),

C₃₃H₄₂F₃N₃O₂ (M=569.71)

Calc.: molecular peak (M+H)⁺: 570

Found: molecular peak (M+H)⁺: 570

### EXAMPLE 17

9-{4-[4-(naphthalen-2-yl-acetyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and naphthalen-2-yl-acetic acid.

Yield: 0.35 g (84% of theory),

C₃₆H₃₁F₃N₃O₂ (M=599.70)

Calc.: molecular peak (M+H)⁺: 600

Found: molecular peak (M+H)⁺: 600

### EXAMPLE 18

9-{4-[4-(biphenyl-4-yl-acetyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and biphenyl-4-yl-acetic acid.

Yield: 0.35 g (80.5% of theory),

C₃₈H₃₈F₃N₃O₂ (M=625.74)

Calc.: molecular peak (M+H)⁺: 626

Found: molecular peak (M+H)⁺: 626

### EXAMPLE 19

9-{4-[4-(1-phenyl-cyclopropanecarbonyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and 1-phenyl-cyclopropanecarboxylic acid.

Yield: 0.2 g (50% of theory),

C₃₄H₃₆F₃N₃O₂ (M=575.68)

Calc.: molecular peak (M+H)⁺: 576

Found: molecular peak (M+H)⁺: 576

### EXAMPLE 20

9-{4-[4-(1-phenyl-cyclopentanecarbonyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and 1-phenyl-cyclopentanecarboxylic acid.

Yield: 0.2 g (43% of theory),

C₃₆H₄OF₃N₃O₂ (M=603.73)

Calc.: molecular peak (M+H)⁺: 604

Found: molecular peak (M+H)⁺: 604

### EXAMPLE 21

9-{4-[4-(4-pyridyl-acetyl)-piperazino]-butyl}-9H-fluorene-9-carboxylic acid-(2,2,2-trifluoro-ethyl)-amide

Prepared analogously to Example 14 from 9-(4-piperazino-butyl)-9H-fluorene-9-carboxylic acid-(2,2,2-trifluoro-ethyl)-amide and 4-pyridylacetic acid.

Yield: 0.15 g (52% of theory),

C₃₁H₃₃F₃N₄O₂ (M=550.62)

Calc.: molecular peak M⁺: 550

Found: molecular peak M⁺: 550

### EXAMPLE 22

9-{4-[4-(benzylcarbamoyl)-piperazino]-butyl}-9H-fluorene-9-carboxylic acid-(2,2,2-trifluoro-ethyl)-amide

A solution of 0.2 g of 9-(4-piperazino-butyl)-9H-fluorene-9-carboxylic acid-(2,2,2-trifluoro-ethyl)-amide in 20 ml of methylene chloride are stirred with 0.062 g of benzyl isocyanate in 5 ml of methylene chloride for 2 hours at ambient temperature. Then the solvent is concentrated by rotary evaporation and the residue is triturated with petroleum ether and dried.

Yield: 0.23 g (88% of theory),

C₃₂H₃₁F₃N₄O₂ (M=564.65)

Calc.: molecular peak (M+H)⁺: 565

Found: molecular peak (M+H)⁺: 565

The following compounds are prepared analogously to Example 22:

(1) 9-(4-{4-[phenylcarbamoyl]-piperazino}-butyl)-9H-fluorene-9-carboxylic acid-(2,2,2-trifluoro-ethyl)-amide

Yield: 94% of theory,

C₃₁H₃₃F₃N₄O₂ (M=550.62)

Calc.: molecular peak (M+H)⁺: 551

Found: molecular peak (M+H)⁺: 551

(2) 9-(4-{4-[4-trifluoro-phenylcarbamoyl]-piperazino}-butyl)-9H-fluorene-9-carboxylic acid-(2,2,2-trifluoro-ethyl)-amide

Yield: 93% of theory,

C₃₂H₃₂F₆N₄O₂ (M=618.62)

Calc.: molecular peak (M+H)⁺: 619

Found: molecular peak (M+H)⁺: 619

(3) 9-(4-{4-[phenylcarbamoyl]-[1.4]diazepan-1-yl}-butyl)-9H-fluorene-9-carboxylic acid-(2,2,2-trifluoro-ethyl)-amide

Yield: 65% of theory,

C₃₂H₃₅F₃N₄O₂ (M=564.65)

Calc.: molecular peak (M+H)⁺: 565

Found: molecular peak (M+H)⁺: 565

### EXAMPLE 23

9-{4-[4-(α, α-dimethyl-3-isopropenyl-benzylcarbamoyl)-piperazino]-butyl}-9H-fluorene-9-carboxylic acid-(2,2,2-trifluoro-ethyl)-amide

Prepared analogously to Example 22 from 9-(4-piperazino-butyl)-9H-fluorene-9-carboxylic acid-(2,2,2-trifluoro-ethyl)-amide and α,α-dimethyl-3-isopropenyl-benzyl isocyanate.

Yield: 0.2 g (82% of theory),

C₃₇H₄₃F₃N₄O₂ (M=632.78)

Calc.: molecular peak (M+H)⁺: 633

Found: molecular peak (M+H)⁺: 633

### EXAMPLE 24

9-(4-{4-[phenyl-acetyl]-2,6-dimethyl-piperazino}-butyl)-9H-fluorene-9-carboxylic acid-(2,2,2-trifluoro-ethyl)-amide

Prepared analogously to Example IV from 9-(4-bromo-butyl)-9H-fluorene-9-carboxylic acid-(2,2,2-trifluoro-ethyl)-amide and 4-phenylacetyl-2,6-dimethylpiperazine.

Yield: 14% of theory,

C₃₄H₃₈F₃N₃O₂ (M=577.69)

Calc.: molecular peak (M+H)⁺: 578

Found: molecular peak (M+H)⁺: 578

The following compounds are prepared analogously to Example 24:

(1) 9-(4-{4-[phenyl-acetyl]-piperazino}-butyl)-9H-fluorene-9-carboxylic acid-propyl-amide

Prepared from 9-(4-bromo-butyl)-9H-fluorene-9-carboxylic acid-propylamide and phenylacetyl-piperazine.

Yield: 48% of theory,

C₃₃H₃₉N₃O₂ (M=509.69)

Calc.: molecular peak (M+H)⁺: 510

Found: molecular peak (M+H)⁺: 510

(2) 9-(4-{4-[phenyl-acetyl]-piperazino}-butyl)-9H-fluorene-9-carboxylic acid-benzyl-amide

Prepared from 9-(4-bromo-butyl)-9H-fluorene-9-carboxylic acid-benzylamide and phenylacetyl-piperazine.

Yield: 54% of theory,

C₃₇H₃₉N₃O₂ (M=557.74)

Calc.: molecular peak (M+H)⁺: 558

Found: molecular peak (M+H)⁺: 558

(3) 9-(4-{4-[phenyl-acetyl]-piperazino}-butyl)-9H-fluorene-9-carboxylic acid-phenyl-amide

Prepared from 9-(4-bromo-butyl)-9H-fluorene-9-carboxylic acid-phenylamide and phenylacetyl-piperazine.

Yield: 55% of theory, C₃₆H₃₇N₃O₂ (M=543.71)

Calc.: molecular peak (M−H)⁻: 542

Found: molecular peak (M−H)⁻: 542

(4) 9-(4-{4-[phenyl-acetyl]-piperazino}-butyl)-9H-fluorene-9-carboxylic acid-cyclopentyl-amide

Prepared from 9-(4-bromo-butyl)-9H-fluorene-9-carboxylic acid-cyclopentylamide and phenylacetyl-piperazine.

Yield: 66% of theory,

C₃₅H₄₁N₃O₂ (M=535.73)

Calc.: molecular peak (M+H)⁺: 536

Found: molecular peak (M+H)⁺: 536

(5) 9-(4-{4-[phenyl-acetyl]-piperazino}-butyl)-9H-fluorene-9-carboxylic acid-N-methyl-N-ethyl-amide

Prepared from 9-(4-bromo-butyl)-9H-fluorene-9-carboxylic acid-N-methyl-N-ethylamide and phenylacetyl-piperazine.

Yield: 30% of theory,

C₃₃H₃₉N₃O₂ (M=509.69)

Calc.: molecular peak (M+H)⁺: 510

Found: molecular peak (M+H)⁺: 510

### EXAMPLE 25

9-[3-(4-phenylacetyl-piperazino)-propyl]-9H-fluorene-9-carboxylic acid-(2,2,2-trifluoro-ethyl)-amide

1.3 g of 9-[3-(4-phenylacetyl-piperazino)-propyl]-9H-fluorene-9-carboxylic acid chloride are dissolved in 20 ml of methylene chloride and at 0° C. added dropwise to a solution of 0.4 g of 2,2,2-trifluoroethylamine-hydrochloride with 0.9 g of triethylamine in 30 ml of methylene chloride. After one hour the mixture is washed with water and the organic phase is concentrated by rotary evaporation. The product is purified by column chromatography on silica gel (dichloromethane/ethanol=19:1).

Yield: 0.8 g (57% of theory),

C₃₁H₃₂F₃N₃O₂ (M=535.62)

Calc.: molecular peak (M+H)⁺: 536

Found: molecular peak (M+H)⁺: 536

### EXAMPLE 26

9-(4-{4-[(4-aminophenyl)-acetyl]-piperazino}-butyl)-9H-fluorene-9-carboxylic acid-(2,2,2-trifluoro-ethyl)-amide

0.5 g of 9-(4-{4-[2-(4-nitrophenyl)-acetyl]-piperazino}-butyl)-9H-fluorene-9-carboxylic acid-(2,2,2-trifluoro-ethyl)-amide are taken up in 20 ml of methanol and hydrogenated over 0.3 g of Raney nickel at ambient temperature and 5 bars of hydrogen pressure for 2.5 h. Then the catalyst is removed by suction filtering and the solution is concentrated by evaporation.

Yield: 95% of theory,

C₃₂H₃₅F₃N₄O₂ (M=564.65)

Calc.: molecular peak (M+H)⁺: 565

Found: molecular peak (M+H)⁺: 565

### EXAMPLE 27

9-(4-{4-[(4-acetylaminophenyl)-acetyl]-piperazino}-butyl)-9H-fluorene-9-carboxylic acid-(2,2,2-trifluoro-ethyl)-amide

0.4 g of 9-(4-{4-[2-(4-aminophenyl)-acetyl]-piperazino}-butyl)-9H-fluorene-9-carboxylic acid-(2,2,2-trifluoro-ethyl)-amide are dissolved in 20 ml of methylene chloride and stirred with 0.1 g of acetylchloride for 1 h at ambient temperature. Then the mixture is washed with water and dilute ammonia and the organic phase is evaporated down.

Yield: 90% of theory,

C₃₄H₃₇F₃N₄O₃ (M=606.69)

Calc.: molecular peak (M+H)⁺: 607

Found: molecular peak (M+H)⁺: 607

### EXAMPLE 28

9-(4-{4-[2-phenylacetyl]-piperazin-2-on-1-yl}-butyl)-9H-fluorene-9-carboxylic acid-(2,2,2-trifluoro-ethyl)-amide

Methyl 9-(4-{4-phenyl-acetyl]-piperazin-2-on-1-yl}-butyl)-9H-fluorene-9-carboxylate is saponified analogously to Example VI and then reacted to form 9-(4-{4-phenyl-acetyl]-piperazin-2-on-1-yl}-butyl)-9H-fluorene-9-carboxylic acid chloride analogously to Example II. 1.5 g of 9-(4-{4-phenyl-acetyl]-piperazin-2-on-1-yl}-butyl)-9H-fluorene-9-carboxylic acid chloride are dissolved in 30 ml of methylene chloride. To this solution are added 0.4 g of 2,2,2-trifluoroethylamine-hydrochloride and 0.9 g of triethylamine in 20 ml of methylene chloride and the mixture is stirred overnight at ambient temperature. Then it is washed with water, the organic phase is concentrated by rotary evaporation and the residue is chromatographed through a silica gel column with methylene chloride/ethanol 20:1.

Yield: 73% of theory,

C₃₂H₃₂F₃N₃O₃ (M=563.62)

Calc.: molecular peak (M+H)⁺: 564

Found: molecular peak (M+H)⁺: 564

The following compounds may be prepared analogously to the foregoing Examples:

(1) 9-[4-(4-phenylacetyl-piperazino)-butyl]-9H-fluorene-9-carboxylic acid-ethylamide

(2) 9-[4-(4-phenylacetyl-piperazino)-butyl]-9H-fluorene-9-carboxylic acid-n-butylamide

(3) 9-[4-(4-phenylacetyl-piperazino)-butyl]-9H-fluorene-9-carboxylic acid-methylamide

(4) 9-[4-(4-phenylacetyl-piperazino)-butyl]-9H-fluorene-9-carboxylic acid-dimethylamide

(5) 9-[4-(4-phenylacetyl-piperazino)-butyl]-9H-fluorene-9-carboxylic acid-N-ethyl-methylamide

(6) 9-[4-(4-phenylacetyl-piperazino)-butyl]-9H-fluorene-9-carboxylic acid-cyclohexylamide

(7) 9-[4-(4-phenylacetyl-piperazino)-butyl]-9H-fluorene-9-carboxylic acid-(2-methoxycarbonyl-ethyl)-amide

(8) 9-[4-(4-phenylacetyl-piperazino)-butyl]-9H-fluorene-9-carboxylic acid-N-methoxycarbonyl-methylamide

(9) 9-(4-{4-[2-phenyl-2-hydroxy-acetyl]-piperazino}-butyl)-9H-fluorene-9-carboxylic acid-(2,2,2-trifluoro-ethyl)-amide

(10) 9-(4-{4-[(4-imidazolyl)-acetyl]-piperazino}-butyl)-9H-fluorene-9-carboxylic acid-(2,2,2-trifluoro-ethyl)-amide

### EXAMPLE 29

Tablets Containing 5 mg of Active Substance Per Tablet

Composition:

| | |
|---|---|
| active substance | 5.0 mg |
| lactose monohydrate | 70.8 mg |
| microcrystalline cellulose | 40.0 mg |
| sodium carboxymethylcellulose, insolubly crosslinked | 3.0 mg |
| magnesium stearate | 1.2 mg |

Preparation:

The active substance is mixed for 15 minutes with lactose monohydrate, microcrystalline cellulose and sodium carboxymethylcellulose in a suitable diffusion mixer. Magnesium stearate is added and mixed with the other substances for another 3 minutes.

The finished mixture is compressed in a tablet press to form facetted flat round tablets.

Diameter of the tablet: 7 mm

Weight of the tablet: 120 mg

### EXAMPLE 30

Capsules Containing 50 mg of Active Substance Per Capsule

Composition:

| | |
|---|---|
| active substance | 50.0 mg |
| lactose monohydrate | 130.0 mg |
| corn starch | 65.0 mg |
| highly dispersed silicon dioxide | 2.5 mg |
| magnesium stearate | 2.5 mg |

Preparation:

A starch paste is prepared by swelling some of the corn starch in a suitable amount of hot water. The paste is then left to cool to room temperature.

The active substance is premixed for 15 minutes in a suitable mixer with lactose monohydrate and corn starch. The starch paste is added and the mixture is mixed with sufficient water to produce a moist homogeneous mass. The moist mass is passed through a screen with a mesh size of 1.6 mm. The screened granules are dried on racks at about 550C for 12 hours.

The dried granules are then passed through screens with mesh sizes of 1.2 and 0.8 mm. Highly dispersed silica is mixed with the granules in a suitable mixer for 3 minutes. Then magnesium stearate is added and mixing is continued for another 3, minutes.

The finished mixture is packed into empty size 1 hard gelatine capsule shells using a capsule filling machine.

### EXAMPLE 31

Tablets Containing 200 mg of Active Substance Per Tablet

Composition:

| | |
|---|---|
| active substance | 200.0 mg |
| lactose-monohydrate | 167.0 mg |
| microcrystalline cellulose | 80.0 mg |
| hydroxypropyl-methylcellulose, type 2910 | 10.0 mg |
| poly-1-vinyl-2-pyrrolidone, insolubly crosslinked | 20.0 mg |
| magnesium stearate | 3.0 mg |

Preparation:

HPMC is dispersed in hot water. After cooling, the mixture yields a clear solution.

The active substance is premixed in a suitable mixer for 5 minutes with lactose monohydrate and microcrystalline cellulose. The HPMC solution is added and the mixing is continued until a homogeneous moist composition is obtained. The moist composition is passed through a screen with a mesh size of 1.6 mm. The screened granules are dried on racks at about 55° C. for 12 hours.

The dried granules are then passed through screens with mesh sizes of 1.2 and 0.8 mm. Poly-1-vinyl-2-pyrrolidone is mixed with the granules in a suitable mixer for 3 minutes. Then magnesium stearate is added and mixing is continued for another 3 minutes.

The finished mixture is compressed in a tablet press to form oblong tablets (16.2×7.9 mm).

Weight of a tablet: 480 mg

1. Substituted piperazine derivatives of general formula 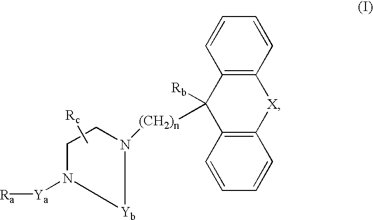
n denotes the number 2, 3, 4 or 5,
X denotes a carbon-carbon bond, an oxygen atom, a methylene, ethylene, imino or N-(C₁₋₃-alkyl)-imino group,
Yₐ denotes a carbonyl or sulphonyl group,
Y_{b} denotes the group —(CH₂)ₘ—, wherein m denotes the number 2 or 3 and wherein a hydrogen atom may be replaced by a C₁₋₃-alkyl group or a methylene group linked to a nitrogen atom may be replaced by a carbonyl group,
Rₐ denotes a C₁₋₆-alkoxy-, phenyl-C₁₋₃-alkoxy or amino group, wherein the amino group may be mono- or disubstituted by C₁₋₃-alkyl-, phenyl-C₁₋₄-alkyl or phenyl groups and the substituents may be identical or different,
a phenyl-, naphthyl, tetrahydronaphthyl, phenoxy or heteroaryl group, a C₁₋₉-alkyl group optionally substituted by a hydroxy, C₁₋₃-alkoxy, C₁₋₄ alkoxycarbonyl or C₁₋₄-alkyl-carbonyloxy group, which may be substituted in the alkyl moiety by a C₁₋₃-alkyl group, by one or two phenyl groups, by a naphthyl, fluorenyl, phenoxy, heteroaryl or C₃₋₇-cycloalkyl group, or a C₃₋₇-cycloalkyl group substituted by a phenyl group,
a phenylcarbonyl, naphthylcarbonyl, tetrahydronaphthylcarbonyl, phenoxycarbonyl or heteroarylcarbonyl group, a C₁₋₉-alkylcarbonyl group, which may be substituted in the alkyl moiety by one or two phenyl groups, by a naphthyl, fluorenyl, phenoxy, heteroaryl or C₃₋₇-cycloalkyl group, or a C₃₋₇-cycloalkylcarbonyl group substituted by a phenyl group,
wherein all the phenyl, naphthyl and heteroaryl moieties mentioned under Rₐ hereinbefore may be substituted by the groups R₁ and R₂, wherein
R₁ denotes a hydrogen, fluorine, chlorine or bromine atom, a cyano, C₁₋₃-alkyl, C₂₋₄-alkenyl, phenyl, hydroxy, C₁₋₄-alkoxy, phenyl-C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl, nitro, amino, C₁₋₃-alkylamino, di-(C₁₋₃-alkyl)-amino, phenyl-C₁₋₃-alkylamino, N-(C₁₋₃-alkyl)-phenyl-C₁₋₃-alkylamino, C₁₋₃-alkylcarbonylamino, N-(C₁₋₃-alkyl) —C₁₋₃-alkylcarbonylamino, C₁₋₃-alkylsulphonylamino or N-(C₁₋₃-alkyl) —C₁₋₃-alkyl-sulphonylamino group and
R₂ denotes a hydrogen, fluorine, chlorine or bromine atom, a C₁₋₃-alkyl, hydroxy or C₁₋₄-alkoxy group, wherein in the abovementioned alkyl and alkoxy moieties of the groups R₁ and R₂ the hydrogen atoms may be wholly or partially replaced by fluorine atoms, or
R₁ and R₂ together denote a methylenedioxy group,
or wherein all the phenyl moieties mentioned above under Rₐ may be substituted by three chlorine or bromine atoms or by three to five fluorine atoms,
R_{b} denotes a carboxy, C₁₋₆-alkoxycarbonyl, C₁₋₆-alkoxycarbonyl-C₁₋₃-alkylcarbonyl, C₃₋₇-cycloalkoxycarbonyl or phenyl-C₁₋₃-alkoxycarbonyl group or a R₃NR₄—CO group wherein
R₃ and R₄, which may be identical or different, denote hydrogen atoms, C₁₋₆-alkyl groups wherein the hydrogen atoms may be wholly or partly replaced by fluorine atoms and the C₁₋₃-alkyl moiety of a C₁₋₃-alkylamino group may be substituted by a carboxy or C₁₋₃-alkoxycarbonyl group or in the 2 or 3 position may also be substituted by an amino, C₁₋₃-alkylamino or di-(C₁₋₃-alkyl)-amino group, C₁₋₃-cycloalkyl, pyridyl, pyridinyl-C₁₋₃-alkyl, phenyl, naphthyl or phenyl-C₁₋₃-alkyl groups, wherein the abovementioned phenyl groups may be substituted in each case by a fluorine, chlorine or bromine atom, by a C₁₋₃-alkyl group wherein the hydrogen atoms may be wholly or partly replaced by fluorine atoms, by a hydroxy, C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl or N,N-di-(C₃₁-alkyl)-amino group, or
R₃ and R₄ together with the nitrogen atom between them denote a 3- to 7-membered cycloalkyleneimino group, wherein the methylene group in the 4 position of a 6 or 7-membered cycloalkyleneimino group may additionally be replaced by an oxygen or sulphur atom, by a sulphinyl, sulphonyl, imino or N-(C₁₋₃-alkyl)-imino group,
and R_{c} denotes a hydrogen atom or a C₁₋₃-alkyl group,
wherein the tricyclic group in the abovementioned general formula I may additionally be mono- or disubstituted by fluorine or chlorine atoms, by methyl or methoxy groups and the substituents may be identical or different,
by the abovementioned heteroaryl groups is meant a 6-membered heteroaryl group, containing one, two or three nitrogen atoms, or
a 5-membered heteroaryl group, containing an imino group optionally substituted by a C₁₋₃-alkyl group, an oxygen or sulphur atom or
an imino group optionally substituted by a C₁₋₃-alkyl group and one or two nitrogen atoms or
an oxygen or sulphur atom and a nitrogen atom,
wherein a phenyl ring may be fused to the above-mentioned heteroaryl groups via a vinylene group,
and wherein the carboxy group mentioned in the definition of the abovementioned groups may be replaced by a group which can be converted into a carboxy group in vivo or by a group which is negatively charged under physiological conditions,
and all the abovementioned saturated alkyl and alkoxy moieties which contain more than 2 carbon atoms may be straight-chain or branched, unless stated otherwise,
the isomers and salts thereof.

2. Substituted piperazine derivatives of general formula I according to claim 1, wherein
X, Yₐ, Y_{b} and Rₐ to R_{c} are defined as in claim 1 and
n denotes the number 3, 4 or 5,
the isomers and salts thereof.

3. Substituted piperazine derivatives of general formula I according to claim 1, wherein
n denotes the number 3 or 4,
X denotes a carbon-carbon bond or an oxygen atom,
Yₐ denotes a carbonyl or sulphonyl group,
Y_{b} denotes the group —(CH₂)ₘ, wherein m denotes the number 2 or 3 and wherein a hydrogen atom may be replaced by a C₁₋₃-alkyl group or a methylene group linked to a nitrogen atom may be replaced by a carbonyl group,
Rₐ denotes a C₁₋₄-alkoxy or phenyl-C₁₋₃-alkoxy group,
an amino group monosubstituted by a C₁₋₃-alkyl, phenyl-C₁₋₃-alkyl or phenyl group or disubstituted by a C₁₋₃-alkyl- and a phenyl-C₁₋₃-alkyl or phenyl group, wherein the alkyl moieties may be straight-chain or branched,
a phenyl, naphthyl, 1,2,3,4-tetrahydro-1-naphthyl, 1,2,3,4-tetrahydro-2-naphthyl, phenoxy or heteroaryl group,
a C₁₋₅-alkyl group,
a C₁₋₃-alkyl group substituted by a C₁₋₇-cycloalkyl, phenyl, phenoxy, 1-naphthyl, 2-naphthyl, fluoren-9-yl or heteroaryl group,
a C₁₋₃-alkyl group disubstituted by two phenyl groups or by a phenyl group and a hydroxy, C₁₋₃-alkoxycarbonyl or C₁₋₃-alkyl-carbonyloxy group,
a C₃₋₇-cycloalkyl group substituted by a phenyl group,
a phenylcarbonyl or naphthylcarbonyl group,
wherein all the phenyl moieties mentioned above under Rₐ may be substituted independently of one another by the groups R₁ and R₂ and all the naphthyl and heteroaryl moieties mentioned above under Rₐ may be substituted by the group R₂, wherein
R₁ denotes a hydrogen, fluorine, chlorine or bromine atom, a cyano, C₁₋₃-alkyl, C₃₋₄-alkenyl, phenyl, hydroxy, C₁₋₃-alkoxy, nitro, amino, C₁₋₃-alkylamino, di-(C₁₋₃-alkyl)-amino, C₁₋₃-alkylcarbonylamino or N-(C₁₋₃-alkyl)-C₁₋₃-alkylcarbonylamino group and
R₂ denotes a hydrogen, fluorine, chlorine or bromine atom, a C₁₋₃-alkyl, hydroxy or C₁₋₃-alkoxy group, wherein in the abovementioned alkyl and alkoxy moieties of the groups R₁ and R2 the hydrogen atoms may be wholly or partially replaced by fluorine atoms, or
R₁ and R₂ together denote a methylenedioxy group,
or wherein all the phenyl moieties mentioned above under Rₐ may be substituted by three chlorine atoms or by three to five fluorine atoms,
R_{b} denotes a C₁₋₃-alkoxycarbonyl, C₁₋₃-alkoxycarbonyl-C₁₋₃-alkylcarbonyl or a R₃NR₄—CO group wherein
R₃ denotes a hydrogen atom or a C₁₋₃-alkyl group and
R₄ denotes a C₁₋₆-alkyl group wherein the hydrogen atoms may be wholly or partly replaced by fluorine atoms, a C₃₋₇-cycloalkyl, phenyl, naphthyl, pyridyl, C₃₋₇-cycloalkyl-C₁₋₃-alkyl, phenyl-C₁₋₃-alkyl or pyridinyl-C₁₋₃-alkyl group,
wherein the abovementioned phenyl groups may be substituted in each case by a fluorine, chlorine or bromine atom, by a C₁₋₃-alkyl group wherein the hydrogen atoms may be wholly or partly replaced by fluorine atoms, or by a hydroxy or C₁₋₃-alkoxy group,
and R_{c} denotes a hydrogen atom or a C₁₋₃-alkyl group,
wherein by the abovementioned heteroaryl group is meant a pyridinyl, pyrazinyl, pyrimidinyl, pyridazinyl, pyrrolyl, furyl, thienyl, oxazolyl, thiazolyl, pyrazolyl, imidazolyl, triazolyl, quinolinyl, quinoxalinyl, quinazolinyl, isoquinolinyl, indolyl or benzimidazolyl group optionally substituted in the carbon skeleton by a C₁₋₃-alkyl group, in which a hydrogen atom bound to a nitrogen atom may be replaced by a C₁₋₃-alkyl group and wherein the 5-membered monocyclic or benzo-condensed heteroaryl groups containing at least one imino group are bound via a carbon or nitrogen atom,
the tricyclic group in the abovementioned general formula I may additionally be substituted by a fluorine or chlorine atom or by a methyl or methoxy group,
and all the abovementioned saturated alkyl and alkoxy moieties which contain more than 2 carbon atoms may be straight-chain or branched, unless stated otherwise,
the isomers and salts thereof.

4. Substituted piperazine derivatives of general formula I according to claim 1, wherein
n denotes the number 4,
X denotes a carbon-carbon bond,
Yₐ denotes a carbonyl group,
Y_{b} denotes the group —(CH₂)₂—,
Rₐ denotes a phenyl-C₁₋₃-alkylamino group,
a straight-chained or branched C₁₋₃-alkyl group substituted by a phenyl or fluoren-9-yl group,
a phenylcarbonyl group,
wherein all the phenyl moieties mentioned above under Rₐ may be substituted independently of one another by the groups R₁ and R₂, wherein
R₁ denotes a hydrogen, fluorine, chlorine or bromine atom, a cyano or C₁₋₃-alkyl group wherein the hydrogen atoms may be wholly or partly replaced by fluorine atoms, and
R₂ denotes a hydrogen, fluorine, chlorine or bromine atom,
R_{b} denotes a R₃NR₄—CO group wherein
R₃ denotes a hydrogen atom and
R₄ denotes a C₁₋₃-alkyl group wherein the hydrogen atoms may be wholly or partly replaced by fluorine atoms, or a phenyl-C₁₋₃-alkyl group,
wherein the abovementioned phenyl groups may in each case be substituted by a fluorine, chlorine or bromine atom, by a C₁₋₃-alkyl group wherein the hydrogen atoms may be wholly or partly replaced by fluorine atoms, by a hydroxy or C₁₋₃-alkoxy group, and
R_{c} denotes a hydrogen atom or a C₁₋₃-alkyl group,
the isomers and salts thereof.

5. The following substituted piperazine derivatives of general formula I according to claim 1:
(1) 9-[4-(4-phenylacetyl-piperazino)-butyl]-9H-fluorene-9-carboxylic acid-(2,2,2-trifluoro-ethyl)-amide,
(2) 9-(4-{4-[2-(4-trifluoromethyl-phenyl)-acetyl]-piperazino}-butyl)-9H-fluorene-9-carboxylic acid-(2,2,2-trifluoro-ethyl)-amide,
(3) 9-{4-[4-(4-bromo-phenylacetyl)-piperazino]-butyl})-9H-fluorene-9-carboxylic acid-(2,2,2-trifluoro-ethyl)-amide
(4) 9-{4-[4-(benzylcarbamoyl)-piperazino]-butyl}-9H-fluorene-9-carboxylic acid-(2,2,2-trifluoro-ethyl)-amide,
(5) 9-(4-{4-[2-phenyl-butyryl]-piperazino}-butyl)-9H-fluorene-9-carboxylic acid-(2,2,2-trifluoro-ethyl)-amide,
(6) 9-[4-(4-chlorophenylacetyl-piperazino)-butyl]-9H-fluorene-9-carboxylic acid-(2,2,2-trifluoro-ethyl)-amide,
(7) 9-(4-{4-[(4-fluorophenyl)-acetyl]-piperazino}-butyl)-9H-fluorene-9-carboxylic acid-(2,2,2-trifluoro-ethyl)-amide,
(8) 9-(4-{4-[phenylacetyl]-piperazino}-butyl)-9H-fluorene-9-carboxylic acid-benzyl-amide,
(9) 9-(4-{4-[(3-chlorophenyl)-acetyl]-piperazino}-butyl)-9H-fluorene-9-carboxylic acid-(2,2,2-trifluoro-ethyl)-amide,
(10) 9-(4-{4-[2-oxo-2-phenyl-acetyl]-piperazino}-butyl)-9H-fluorene-9-carboxylic acid-(2,2,2-trifluoro-ethyl)-amide,
(11) 9-(4-{4-[(2,4-dichlorophenyl)-acetyl]-piperazino}-butyl)-9H-fluorene-9-carboxylic acid-(2,2,2-trifluoro-ethyl)-amide,
(12) 9-(4-{4-[(2,3-difluorophenyl)-acetyl]-piperazino}-butyl)-9H-fluorene-9-carboxylic acid-(2,2,2-trifluoro-ethyl)-amide,
(13) 9-(4-(4-[(fluoren-9-yl)-acetyl]-piperazino}-butyl)-9H-fluorene-9-carboxylic acid-(2,2,2-trifluoro-ethyl)-amide,
(14) 9-(4-}4-[(2,4-dichlorophenyl)-acetyl]-(S)-2-methyl-piperazino}-butyl)-9H-fluorene-9-carboxylic acid-(2,2,2-trifluoro-ethyl)-amide and
(15) 9-(4-{4-[(2,4-dichlorophenyl)-acetyl]-(R)-2-methyl-piperazino}-butyl)-9H-fluorene-9-carboxylic acid-(2,2,2-trifluoro-ethyl)-amide,
and the salts thereof.

6. Physiologically acceptable salts of the compounds according to claims 1 to 5.

7. Medicaments, containing a compound according to at least one of claims 1 to 5 or a salt according to claim 6 optionally together with one or more inert carriers and/or diluents.

8. Use of a compound according to at least one of claims 1 to 5 or a salt according to claim 6 for the preparation of a medicament having a lowering effect on the plasma levels of atherogenic lipoprotein.

9. Process for preparing a medicament according to claim 7, characterised in that a compound according to at least one of claims 1 to 5 or a salt according to claim 6 is incorporated in one or more inert carriers and/or diluents by a non-chemical method.

10. Process for preparing the compounds according to claims 1 to 6, characterised in that
a. a compound of general formula 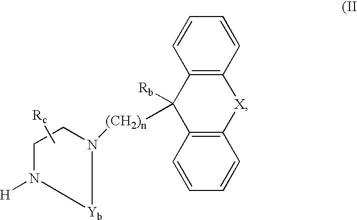
wherein
R_{b}, R_{c}, X, Y_{b} and n are defined as in claims 1 to 5, is reacted with a compound of general formula
Rₐ—Yₐ-Z₁, (III)
wherein
Rₐ and Yₐ are as hereinbefore defined and
Z₁ denotes a hydroxy or nucleofugic leaving group or, if Yₐ denotes a carbonyl group, Z₁together with the hydrogen atom of an adjacent NH group in the group Rₐ denotes another carbon-nitrogen bond, or
b. in order to prepare a compound of general formula I wherein R_{b} denotes a C₁₋₆-alkoxycarbonyl, C₃₋₇-cycloalkoxycarbonyl or phenyl-C₁₋₃-alkoxycarbonyl group or a R₃NR₄—CO group wherein R₃ and R₄ are defined as in claims 1 to 5, a compound of general formula 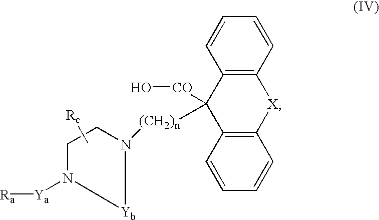
Rₐ, R_{c}, X, Yₐ, Y_{b} and n are defined as in claims 1 to 5, is reacted with a compound of general formula
H—R_{b}′, (V)
wherein
R_{b}′ denotes a C₁₋₆-alkoxy, C₃₋₇-cycloalkoxy or phenyl-C₁₋₃-alkoxy group or a R₃NR₄ group, wherein R₃ and R₄ are defined as in claims 1 to 5, or with the reactive derivatives thereof, and
if desired a compound of general formula I thus obtained which contains an amino or alkylamino group is converted by acylation into a corresponding acyl compound and/or
a compound of general formula I thus obtained which contains a nitro group is converted by reduction into a corresponding amino compound, and/or
if necessary, any protecting group used during the reactions to protect reactive groups is cleaved and/or
a compound of general formula I thus obtained is resolved into the stereoisomers thereof and/or
a compound of general formula I thus obtained is converted into the salts thereof, particularly for pharmaceutical use into the physiologically acceptable salts thereof with an inorganic or organic acid or base.
